(19) United States
(12) Patent Application Publication
(10) Pub. No.: US 2016/0150948 A1
(43) Pub. Date: Jun. 2, 2016

(54) OPTICAL SCANNING UNIT, OPTICAL SCANNING OBSERVATION APPARATUS, AND OPTICAL FIBER SCANNING APPARATUS

(71) Applicant: OLYMPUS CORPORATION, Tokyo (JP)

(72) Inventor: Atsuyoshi SHIMAMOTO, Tokyo (JP)

(73) Assignee: OLYMPUS CORPORATION, Tokyo (JP)

(21) Appl. No.: 14/947,322

(22) Filed: Nov. 20, 2015

Related U.S. Application Data
(63) Continuation of application No. PCT/JP2014/002673, filed on May 21, 2014.

(30) Foreign Application Priority Data
May 21, 2013 (JP) 2013-107288
Nov. 26, 2013 (JP) 2013-2444009

(51) Int. Cl. A61B 1/07; A61B 1/06; A61B 1/04; A61B 1/00

(52) U.S. Cl. CPC A61B 1/07; A61B 1/00009; A61B 1/00006; A61B 1/0638; A61B 1/0676; A61B 1/00158; A61B 1/04; A61B 1/00096

(57) ABSTRACT

Provided is an optical scanning unit including: an optical fiber; an actuator; a reducing part; a detection part; and a controller. The optical fiber oscillates the emitting end, to thereby scan the object. The actuator oscillates the emitting end. The reducing part reduces the transmission of light emitted from the emitting end. The detection part detects light at the object when light is irradiated onto the object. The controller forms an image, based on light detected by the detection part and the state of oscillation of the optical fiber. The controller calculates the eccentricity in the image thus formed, based on a position where the received amount of light is reduced and the center position of the image.

### CROSS REFERENCE TO RELATED APPLICATIONS

The present application is a Continuing Application based on PCT/JP2014/002673 filed on May 21, 2014, which, in turn, claims the priority from Japanese Patent Application No. 2013-107288 filed on May 21, 2013, and Japanese Patent Application No. 2013-244009 filed on Nov. 26, 2013, the entire disclosures of which is incorporated herein by reference.

### TECHNICAL FIELD

The present disclosure relates to an optical scanning unit, an optical scanning observation apparatus, and an optical fiber scanning apparatus, each for calculating the eccentricity between the designed optical axis of an optical fiber when not oscillated and the actual optical axis thereof.

### BACKGROUND

There has been known an optical scanning unit in which an optical fiber for emitting light is oscillated, to thereby scan an observation object so as to allow an image thereof to be captured (see Patent Literature 1 (PTL 1)), or to thereby scan an irradiation surface so as to allow an image to be formed.

The optical scanning unit generally employs an illumination optical system having a short focal length disposed on the object side to be irradiated with light, relative to the emitting end of the optical fiber. With the short focal length of the illumination optical system, even a slightest eccentricity of the optical fiber from a designed installation position thereof could lead to a significant deviation of the irradiation region of light on the object. For this reason, the optical fiber needs to be installed with strict accuracy, which has been in need of complicated and skilled processes such as, for example, complicated operations with the use of a precision stage.

Further, there has conventionally known a fiber scanning observation apparatus in which light is scanned from the emitting end of an optical fiber toward an object, so as to detect light reflected and scattered by the object or fluorescence generated in the object (see, for example, PTL 2). In such apparatus, in order to scan irradiated light on the object, the tip end part of the optical fiber is cantilevered with the emitting end for emitting light being oscillatable, and a drive mechanism such as a piezoelectric element is arranged in the vicinity of the cantilevered part, to thereby vibrate the optical fiber.

Examples of a generally-known scanning method for an optical fiber may include: a spiral scan where the spot of irradiated light is scanned to draw a spiral; and a raster scan where the optical fiber is vibrated at high speed in one direction while being moved at slower speed in a direction orthogonal thereto. In the raster scan, the vibration frequency is generally set equal to or approximately equal to the resonance frequency. Further, in the raster scan, the optical fiber may preferably be vibrated in the vicinity of a resonance frequency, in the direction of high speed vibration. Accordingly, it has been hitherto practiced to vibratory drive the fiber based on a resonance frequency determined based on the design value of the optical fiber scanning apparatus.

Further, in the optical scanning apparatus, there may be used a device such as a sensor for detecting the position of the fiber, so as to acquire in advance the coordinate data on the irradiation position of light from the optical fiber, as a function of time from the start of scan. In scanning the actual object, a pixel signal detected according to the time from the start of scan is mapped on the two-dimensional coordinate, to thereby generate an image.

However, the properties of the optical fiber (such as Young's modulus or density) may not always be constant, but rather change over time due to the ambient environmental change such as temperature variation, the change of the constituent elements over time, and an impact between the fiber and the object when in use. Further, properties of members such as a piezoelectric element constituting the drive mechanism may also change over time. When the optical fiber or of the drive mechanism suffer characteristic change over time, the resonance frequency at the tip end part of the optical fiber, the Q value of the vibration, and the drive power of the drive mechanism would vary. As a result, the scanning trajectory of the optical fiber will deviate from the originally-expected scanning trajectory, which will be illustrated with reference to FIGS. 38, 39.

FIG. 38 each show, as a simplified example, a scanning of an optical fiber along a circular trajectory, where: FIG. 38A shows a trajectory of the tip of an optical fiber in the X direction; and FIG. 38B shows a trajectory of the tip of an optical fiber in the Y direction. Further, FIG. 38C shows the trajectory of optical scanning in the XY plane. The vibration of the fiber tip in the X direction is shifted in phase by 90 degrees from the vibration of the fiber tip in the Y direction, and thus the fiber tip draws a circular orbit. Meanwhile, FIG. 39 each show the scanning of an optical fiber having been changed over time. Changes in the resonance frequency, the Q value of the vibration, and the drive power of the drive mechanism will change the phase and the amplitude, as can be seen in the trajectory of the fiber tip in the X direction and Y direction of FIGS. 39A and 39B. Accordingly, as illustrated in FIG. 39C, the trajectory of the optical scanning on the object also suffers deformation.

The above description applies to a case of a circular orbit. However, in the case of spiral scan for example, a scanning trajectory 1011 suffers deformation, as indicated by the solid line of FIG. 40, from an originally-expected trajectory (broken line) 1021. When the trajectory of optical scanning is deformed as described above, an image of the object formed by mapping pixel data on a two-dimensional coordinate based on the previously-expected trajectory of the optical fiber will be distorted and made different from the actual state. In order to overcome such problem, according to the invention of PTL 2, a calibration image pattern is provided so as to actually acquire the image and compare the acquired image with a previously-stored calibration image, to thereby calculate the distortion of the trajectory so as to carry out the process of compensating the distortion.

However, according to the method of PTL 2, it is necessary to prepare a calibration image pattern and to acquire the same as an image. This may be effective in acquiring compensation data in shipment from factory. However, when the properties of the optical fiber and the drive mechanism have changed after once the trajectory is compensated, the observation must be stopped to again carry out the calibration, which means that the calibration cannot be effected during the observation of the object. For example, when the properties have changed during an endoscopic intracorporeal observation, the observation apparatus needs to be temporarily taken out of the body, which requires complicated operations.

### CITATION LIST

### Patent Literature

PTL 1: JP 2010-142482 A

PTL 2: JP 2010-515947 A

### SUMMARY

An optical scanning unit according to a first one of the disclosed aspects includes:

an optical fiber having an emitting end thereof oscillatably supported, the emitting end being oscillated while irradiating an object with light emitted from the emitting end, to thereby scan the object;

an actuator for oscillating the emitting end;

a reducing part disposed at a predetermined position relative to a designed optical axis of the optical fiber when not oscillated, the predetermined position being on the object side relative to the emitting end, the reducing part reducing the transmission of light having at least part of the bandwidth of light emitted from the emitting end;

a detection part for detecting light at the object when light emitted from the emitting end is irradiated onto the object; and

a controller for calculating, in an image formed based on the light detected by the detection part and the state of oscillation of the optical fiber, the eccentricity between the designed optical axis and the actual optical axis of the optical fiber, based on a position where the received amount of the light having part of the bandwidth is reduced and the center position of the image.

In the optical scanning unit according to a second one of the disclosed aspects,

the reducing part may preferably reduce the transmission of at least any of light of three colors minimally necessary for forming the image as a color image.

In the optical scanning unit according to a third one of the disclosed aspects,

the controller may preferably calculate the eccentricity in an eccentricity calculation frame.

In the optical scanning unit according to a fourth one of the disclosed aspects,

the eccentricity calculation frame may preferably be carried out at least any one of: in between main purpose frames for scanning the object for a different purpose rather than to calculate the eccentricity; and before or after an operation mode for scanning the object for the different purpose.

In the optical scanning unit according to a fifth one of the disclosed aspects, the reducing part may preferably transmit light of three colors necessary for forming the image as a color image, and reduce transmission of light of different bandwidth from the light of three colors.

In the optical scanning unit according to a sixth one of the disclosed aspects,

the reducing part may preferably substantially shield light having at least part of the bandwidth of light emitted from the emitting end.

In the optical scanning unit according to a seventh one of the disclosed aspects,

the reducing part may preferably reduce the transmission of the light having part of the bandwidth, in a region capable of passing or transmitting light.

In the optical scanning unit according to an eighth one of the disclosed aspects,

the reducing part may preferably be in a shape that surrounds around the region capable of passing or transmitting light.

The optical scanning unit according to a ninth one of the disclosed aspects, may preferably further include an illumination optical system disposed in the emission direction of the emitting end,

in which the reducing part may preferably be disposed on a mirror frame holding the illumination optical system.

The optical scanning unit according to a tenth one of the disclosed aspects, may preferably further include an illumination optical system disposed in the emission direction of the emitting end,

in which the reducing part may preferably formed as a film for reducing the transmission of the light having part of the bandwidth, the film being disposed on a surface of an optical element included in the illumination optical system.

In the optical scanning unit according to an eleventh one of the disclosed aspects,

the region capable of passing or transmitting light within the reducing part may preferably be circular or rectangular in shape.

In the optical scanning unit according to a twelfth one of the disclosed aspects,

the controller may preferably cause:

the actuator to oscillate the emitting end such that, when calculating the eccentricity, light emitted from the emitting end is irradiated onto a region larger than the inside of the reducing part; and

the actuator to oscillate the emitting end such that, at times other than calculating the eccentricity, light emitted from the emitting end is irradiated onto a region smaller than the inside of the reducing part.

The optical scanning unit according to a thirteenth one of the disclosed aspects, may preferably further includes an illumination optical system disposed in the emission direction of the emitting end,

in which the reducing part may preferably be disposed on the object side relative to the incident pupil position of the illumination optical system.

In the optical scanning unit according to a fourteenth one of the disclosed aspects,

the controller may preferably oscillate the emitting end in a state where the eccentricity of the optical fiber has been compensated based on the eccentricity calculated.

In the optical scanning unit according to a fifteenth one of the disclosed aspects,

the actuator may be a piezoelectric actuator or an electromagnetic actuator.

An optical scanning observation apparatus according to a sixteenth one of the disclosed aspects includes: an optical scanning unit having:

an optical fiber having an emitting end oscillatably supported, the emitting end being oscillated while irradiating an object with light emitted from the emitting end, to thereby scan the object;

an actuator for oscillating the emitting end;

a reducing part disposed at a predetermined position relative to a designed optical axis of the optical fiber when not oscillated, the predetermined position being on the object side relative to the emitting end, the reducing part reducing the transmission of light having at least part of the bandwidth of light emitted from the emitting end;

a detection part for detecting light at the object when light emitted from the emitting end is irradiated onto the object; and

a controller for calculating, in an image formed based on the light detected by the detection part and the state of oscillation of the optical fiber, the eccentricity between the designed optical axis and the actual optical axis of the optical fiber, based on a position where the received amount of the light having a part of the bandwidth is reduced and the center position of the image.

An optical fiber scanning apparatus according to a seventeenth one of the disclosed aspects, includes:

an optical fiber for guiding light from a light source and irradiating the light onto an object;

an actuator for vibratory driving a tip end part of the optical fiber;

a light reducing part for partially reducing transmission of light having at least part of the bandwidth of light emitted from an emitting end of the tip end part;

a detection part for detecting light to be detected, the light being obtained from the object, through irradiation of light emitted from the emitting end; and

a controller for controlling the vibratory-drive of the actuator,

in which the controller identifies, based on a signal detected by the detection part, timing for reducing, by the light reducing part, the transmission of the light having at least part of the bandwidth.

In the optical fiber scanning apparatus according to an eighteenth one of the disclosed aspects,

the controller may preferably calculate, based on the timing for reducing the transmission of the light having at least part of the bandwidth, the amplitude of the tip end part of the optical fiber.

In the optical fiber scanning apparatus according to a nineteenth one of the disclosed aspects,

the actuator may preferably sequentially change, within a predetermined frequency range, the drive frequency for vibratory driving the tip end part of the optical fiber, and

the controller may preferably calculate, based on the drive frequency and the calculated amplitude of the tip end part, the resonance frequency of the tip end part of the optical fiber.

In the optical fiber scanning apparatus according to a twentieth one of the disclosed aspects,

the controller may preferably calculate, based on the drive frequency in sequence and the calculated amplitude of the tip end part, the Q value of the vibration of the tip end part of the optical fiber.

In the optical fiber scanning apparatus according to a twenty-first one of the disclosed aspects,

the actuator may preferably be capable of vibratory driving the tip end part of the optical fiber individually in at least two driving directions, and the controller may preferably calculate, for each of the at least two driving directions, the amplitude of the tip end part of the optical fiber.

In the optical fiber scanning unit according to a twenty-second one of the disclosed aspects,

the controller may preferably calculate the amplitude of the drive signal to be applied to the actuator such that the time interval of the timing for reducing the transmission of the light having at least part of the bandwidth is made coincide with a predetermined time interval.

In the optical fiber scanning unit according to a twenty-third one of the disclosed aspects,

the controller may preferably change, based on information obtained from the timing for reducing the transmission of the light having at least part of the bandwidth, either one or both of the amplitude and the phase of the drive signal to be applied to the actuator, so as to keep constant the trajectory of the emitting end of the optical fiber.

The optical fiber scanning unit according to a twenty-fourth one of the disclosed aspects, may preferably further include an image acquisition part for acquiring image data on the object, and have a vibration adjustment mode and an image acquisition mode,

in which, in the vibration adjustment mode, the controller may preferably vibratory drive the actuator, and calculate, based on the timing for reducing the transmission of the light having at least part of the bandwidth, a correction value of the drive parameter of the actuator including at least one of the amplitude and the phase of the drive signal to be applied to the actuator in order for obtaining a predetermined vibration trajectory, and in the image acquisition mode, the controller may preferably vibratory drive the actuator based on the correction value of the drive parameter, and the image acquisition part may preferably acquire image data on the object, from the signal detected by the detection part.

In the optical fiber scanning unit according to a twenty-fifth one of the disclosed aspects,

the vibration adjustment mode may preferably be carried out, for each image acquisition for one frame in the image acquisition mode, before and after the image for one frame is acquired.

In the optical fiber scanning unit according to a twenty-sixth one of the disclosed aspects,

the vibration adjustment mode may preferably be carried out after activation of the scanning detection apparatus and before executing the image acquisition mode.

The optical fiber scanning unit according to a twenty-seventh one of the disclosed aspects, may preferably further include an optical system for irradiating, toward the object, light emitted from the emitting end of the optical fiber,

in which the light reducing part may preferably be provided to a mirror frame holding the optical system.

The optical fiber scanning unit according to a twenty-eighth one of the disclosed aspects, may preferably further include an optical system for irradiating, toward the object, light emitted from the emitting end of the optical fiber,

in which the light reducing part may preferably be formed on a surface of an optical element constituting the optical system.

In the optical fiber scanning unit according to a twenty-ninth one of the disclosed aspects,

the light reducing part may preferably be selected so as to reduce the transmission of light having a wavelength that does not coincide with the absorption wavelength of the object.

An optical fiber scanning apparatus according to a thirtieth one of the disclosed aspects includes:

an optical fiber having a tip end part oscillatably supported, the optical fiber guiding light from a light source part to irradiate an object with the light;

an actuator for vibratory driving the tip end part of the optical fiber;

a light reducing part for partially reducing transmission of light having at least part of the bandwidth of light emitted from an emitting end of the tip end part;

a detection part for detecting light to be detected, the light being obtained from the object through irradiation of light emitted from the emitting end; and

a controller for controlling the vibratory-drive of the actuator,

wherein the controller identifies, based on a signal detected by the detection part, timing for reducing, by the light reducing part, the transmission of the light having at least part of the bandwidth, and calculates, based on the timing, a vibration period of each vibration of the tip end part of the optical fiber.

In the optical fiber scanning apparatus according to a thirty-first one of the disclosed aspects,

the controller may preferably vibratory drive the actuator in a two-dimensional direction so as to two-dimensionally scan the object.

In the optical fiber scanning apparatus according to a thirty-second one of the disclosed aspects,

the two-dimensional scan may preferably be spiral scan.

In the optical fiber scanning apparatus according to a thirty-third one of the disclosed aspects,

the light reducing part may preferably be disposed so as to radially traverse, on the optical path of the spiral scan, a region that does not include the scan center and an outermost circumferential part of the spiral scan, and the controller may preferably calculate, based on the timing for reducing the transmission of the light having at least part of the bandwidth, the amplitude of the tip end part of the optical fiber.

The optical fiber scanning apparatus according to a thirty-fourth one of the disclosed aspects, may preferably further include an image acquisition part for acquiring image data on the object,

in which the image acquisition part may preferably acquire image data on the object from the signal detected by the detection part, through each scan of the object, and the controller may preferably calculate the period and the amplitude of each vibration of the tip end part, and adjusts, based on the amplitude and the period thus calculated, a drive signal to be applied to the actuator.

### BRIEF DESCRIPTION OF THE DRAWINGS

In the accompanying drawings:

FIG. 1 is a functional block diagram schematically illustrating an internal configuration of an optical scanning observation apparatus having an optical scanning unit according to Embodiment 1 of the present disclosure;

FIG. 2 is a functional block diagram schematically illustrating an internal configuration of the light source part of Embodiment 1;

FIG. 3 is a schematic overview of the optical scanning endoscope main body of FIG. 1;

FIG. 4 is an enlarged sectional view of the tip end part of the optical scanning endoscope main body according to Embodiment 1;

FIG. 5 is an enlarged perspective view of the proximity of the actuator of FIG. 4;

FIG. 6 is a functional block diagram schematically illustrating an internal configuration of the detection part according to Embodiment 1;

FIG. 7 is a graph showing a waveform of a drive signal for calculating the eccentricity, generated in Embodiment 1;

FIG. 8 is a partial-enlarged view of the tip end part for illustrating the field angle in the optical scanning endoscope main body of FIG. 1;

FIG. 9 is a schematic view of an image for calculating the eccentricity according to Embodiment 1;

FIG. 10 is a graph showing a waveform of an observation drive signal generated in Embodiment 1;

FIG. 11 is a flowchart for illustrating an eccentricity calculation process to be carried out by the controller in Embodiment 1;

FIG. 12 is a flowchart for illustrating an observation process to be carried out by the controller in Embodiment 1;

FIG. 13 is a functional block diagram schematically illustrating an internal configuration of the light source part according to Embodiment 2 of the present disclosure;

FIG. 14 is an enlarged sectional view of the tip end part of the optical scanning endoscope main body according to Embodiment 2;

FIG. 15 is a front view of the plate of FIG. 14;

FIG. 16 is a functional block diagram schematically illustrating an internal configuration of the detection part according to Embodiment 2;

FIG. 17 is a schematic view of an image for calculating the eccentricity, according to Embodiment 2;

FIG. 18 is a flowchart for illustrating an observation process to be carried out by the controller in Embodiment 2;

FIG. 19 is a block diagram illustrating a schematic configuration of a fiber scanning endoscope apparatus as an example of the optical fiber scanning apparatus according to Embodiment 3 of the present disclosure;

FIG. 20 is a schematic external view of the scope of the fiber scanning endoscope apparatus of FIG. 19;

FIG. 21 is a sectional view of the tip end part of the scope of FIG. 20;

FIG. 22 illustrates a configuration of the optical system of FIG. 21;

FIG. 23 illustrates the light shielding mask disposed in the optical system of FIG. 22;

FIG. 24A is a perspective view of the actuator located at the tip end part of the scope of FIG. 21;

FIG. 24B is a sectional view taken along a plane perpendicular to the axis of the optical fiber of the actuator of FIG. 24A;

FIG. 25 is a flowchart illustrating an image acquisition procedure in Embodiment 3;

FIG. 26A illustrates a light shielding region in the X direction and a scanning range on the light shielding mask, for illustrating an example where the light shielding mask is used to optically scan an object at a predetermined amplitude before the change over time;

FIG. 26B is a graph showing a temporal change of signal strength of detected near-infrared light, for illustrating an example where the light shielding mask is used to optically scan an object at a predetermined amplitude before the change over time;

FIG. 27A illustrates a light shielding region in the X direction and a scanning range on the light shielding mask, for illustrating an example where the light shielding mask is used to optically scan an object at a narrowed amplitude;

FIG. 27B is a graph showing a temporal change of signal strength of detected near-infrared light, for illustrating an example where the light shielding mask is used to optically scan an object at a narrowed amplitude;

FIG. 28 is a graph for illustrating a method for detecting a resonance frequency and a Q value;

FIG. 29 is a graph showing a relation between the frequency of an optical fiber and the phase delay of the fiber;

FIG. 30 is a graph for illustrating a relation between the frequency and the amplitude of an optical fiber;

FIG. 31 is a flowchart illustrating an image acquisition procedure according to Embodiment 4 of the present disclosure;

FIG. 32 is for illustrating a light shielding mask according to Embodiment 5 of the present disclosure and a scanning trajectory on the light shielding mask;

FIGS. 33A and 33B are graphs for illustrating a method for analyzing the period and the amplitude for each round of spiral scan using the light shielding mask of FIG. 32;

FIG. 34A shows a variation of the light shielding mask, which has a light shielding pattern where dot-shaped light shielding regions are distributed;

FIG. 34B shows another variation of the light shielding mask, which has a light shielding pattern with a plurality of linear-shaped light shielding regions;

FIG. 34C shows still another variation of the light shielding mask, which has a light shielding pattern with a semicircular light shielding region;

FIG. 35 is a front view of a mirror frame and a lens located at the scope tip of a fiber scanning endoscope apparatus according to Embodiment 6 of the present disclosure;

FIG. 36 is a graph showing a temporal change of signal light strength when the mirror frame of FIG. 35 is used;

FIG. 37A is a side view of an exemplary actuator which is configured by using a piezoelectric element;

FIG. 37B is a sectional view taken along the line A-A of FIG. 37A;

FIG. 38A shows a trajectory of the tip of an optical fiber in the X direction, when the optical fiber is scanned along a circular orbit;

FIG. 38B shows a trajectory of the tip of an optical fiber in the Y direction, when the optical fiber is scanned along a circular orbit;

FIG. 38C shows a trajectory of optical scanning in the XY plane, when the optical fiber is scanned along a circular orbit;

FIG. 39A shows a trajectory of the tip of an optical fiber in the X direction, in an exemplary case where the scanning trajectory of FIG. 38 has changed over time;

FIG. 39B shows a trajectory of the tip of an optical fiber in the Y direction, in an exemplary case where the scanning trajectory of FIG. 38 has changed over time;

FIG. 39C shows a trajectory of the optical scanning in the XY plane, in an exemplary case where the scanning trajectory of FIG. 38 has changed over time; and

FIG. 40 illustrates an example of a deformed trajectory of spiral scan.

### DETAILED DESCRIPTION

Hereinafter, Embodiments of the present disclosure will be illustrated with reference to the accompanying drawings.

### Embodiment 1

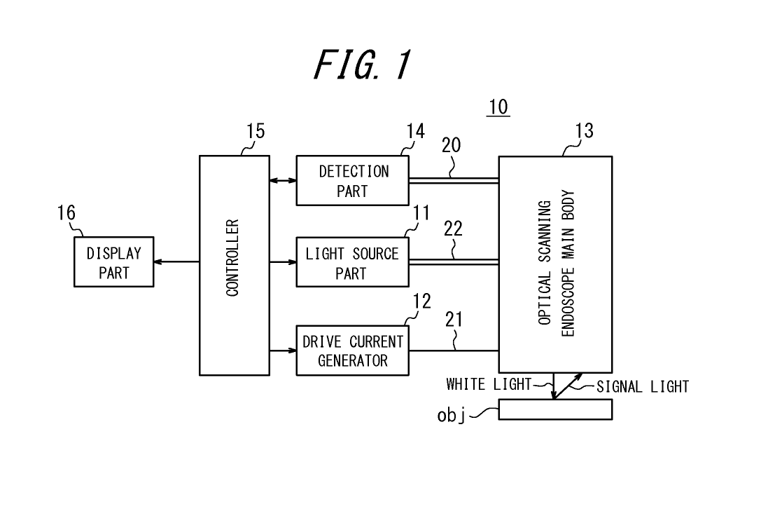

FIG. 1 is a functional block diagram schematically illustrating an internal configuration of an optical scanning observation apparatus having an optical scanning unit according to Embodiment 1 of the present disclosure.

The optical scanning observation apparatus 10 is, for example, an optical scanning endoscope apparatus, and configured by including: a light source part 11; a drive current generator 12; an optical scanning endoscope main body 13; a detection part 14; a controller 15: and a display part 16.

The light source part 11 emits white light, which is supplied to the optical scanning endoscope main body 13. The drive current generator 12 transmits, to the optical scanning endoscope main body 13, a drive signal necessary for scanning an object obj. The optical scanning endoscope main body 13 scans the object obj with the white light thus supplied, so as to propagate signal light, which has been obtained through the scan, to the detection part 14. The detection part 14 converts the signal light thus propagated into an electric signal. The controller 15 synchronously controls the light source part 11, the drive current generator 12, and the detection part 14, while processing the electric signal output from the detection part 14 to synthesize an image so as to display the image on the display part 16.

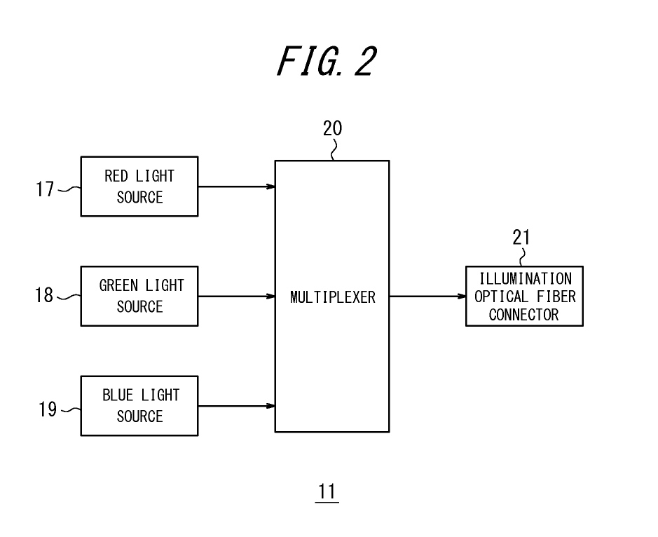

The light source part 11 is configured by including, as illustrated in FIG. 2: a red light source 17; a green light source 18; a blue light source 18; a multiplexer 20; and an illumination optical fiber connector 21. The red light source 17 may be a red laser, which emits red light of 640 nm in wavelength. The green light source 18 may be a green laser, which emits green light of 532 nm in wavelength. The blue light source 19 may be a blue laser, which emits blue light of 445 nm in wavelength. The multiplexer 20 may include, for example, a dichroic mirror and a fiber combiner, and multiplexes red light, green light, and blue light each radiated by the red light source 17, the green light source 18, and the blue light source 19, respectively. The illumination optical fiber connector 21 is optically connected to an illumination optical fiber 22 provided to the optical scanning endoscope main body 13, and supplies the illumination optical fiber 22 with white light multiplexed by the multiplexer 20.

The drive current generator 12 (see FIG. 1) generates, based on the control of the controller 15, a drive signal that displaces the emitting end of the illumination optical fiber 22 in a swirling manner. The drive current generator 12 supplies the drive signal to an actuator provided to the optical scanning endoscope main body 13.

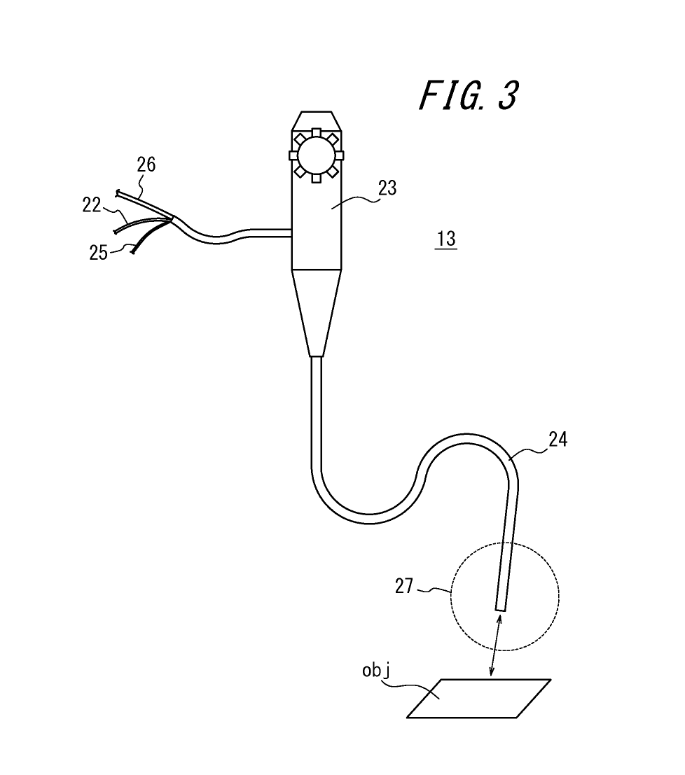

The optical scanning endoscope main body 13 includes, as illustrated in FIG. 3, an operation portion 23 and an insertion portion 24, where one end of the operation portion 23 is connected to the base end of the insertion portion 24 so as to be integrally formed together.

The optical scanning endoscope main body 13 is configured by including: the illumination optical fiber 22; a wiring cable 25; and a detection optical fiber bundle 26. The illumination optical fiber 22, the wiring cable 25, and the detection optical fiber bundle 26 are guided through the inside the insertion portion 24 from the operation portion 23, to the tip end part 27 (circled by the broken line of FIG. 3) of the insertion portion 24. The illumination optical fiber 22 is connected, on the operation portion 23 side, to an illumination optical fiber connector 21 of the light source part 11, so as to propagate the white light to the tip end part 27. The wiring cable 25 is connected, on the operation portion 23 side, to the drive current generator 12, and transmits a drive signal to the actuator disposed in the tip end part 27. The detection optical fiber bundle 26 is connected, on the operation portion 23 side, to the detection part 14, and propagates signal light obtained at the tip end part 27 to the detection part 14.

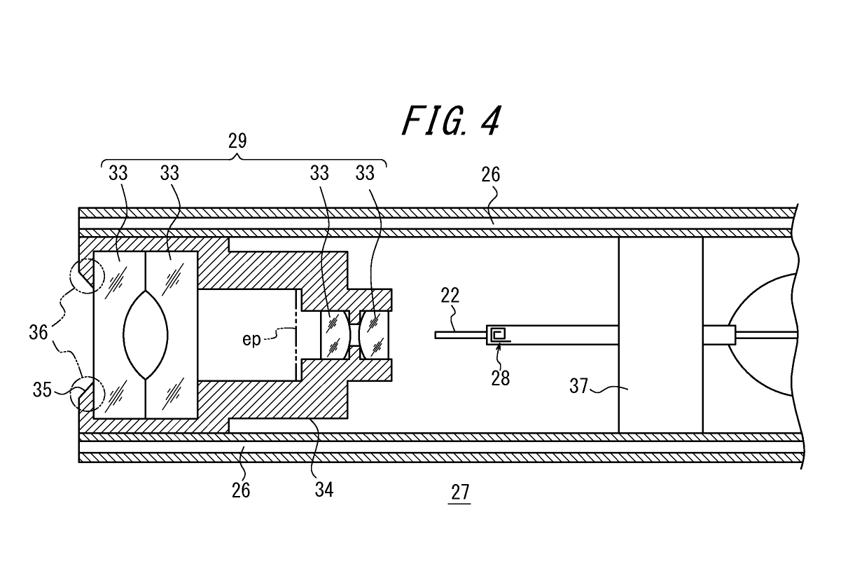

FIG. 4 is an enlarged sectional view of the tip end part 27 of the optical scanning endoscope main body 13 of FIG. 1. The tip end part 27 includes an actuator 28, an illumination optical system 29, and a detection lens (not shown), while having the illumination optical fiber 22 and the detection optical fiber bundle 26 extending therethrough.

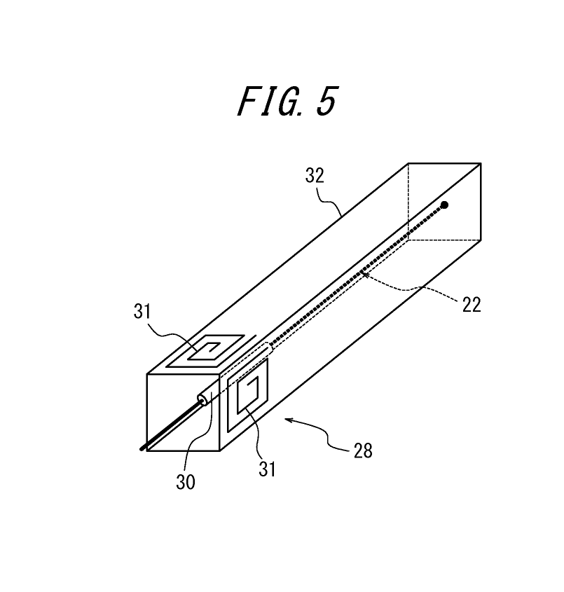

The actuator 28 is, for example, an electromagnetic actuator, and includes a permanent magnet 30 (see FIG. 5) and a deflection magnetic field generating coil 31. The permanent magnet 30 is cylindrical in shape, and is attached to the illumination optical fiber 22, with the illumination optical fiber 22 being inserted therethrough. The illumination optical fiber 22 is supported by an angular tube 32, so as to be oscillatable in the vicinity of the emitting end including the permanent magnet 30. The deflection magnetic field generating coil 31 is disposed on four faces of the angular tube 32. The angular tube 32 employed in Embodiment 1 is in a rectangular column shape, but may be in a cylindrical shape or other shape that is hollow inside. The deflection magnetic field generating coil 31 generates a magnetic field based on a drive signal supplied by the drive current generator 12, and deflects the emitting end of the illumination optical fiber 22 together with the permanent magnet 30 along two directions. The actuator 28 vibrates the emitting end of the illumination optical fiber 22 based on the drive signal so as to deflect the emitting end such that the amplitude is increased from zero to the maximum amplitude and then reduced to zero again, within one frame. The actuator 28 vibrates the emitting end of the illumination optical fiber 22 as described above along two different directions, to thereby spirally scan the object obj with white light emitted from the emitting end.

The illumination optical system 29 (see FIG. 4) is disposed at the extreme tip of the tip end part 27 of the insertion portion 24, that is, in the emission direction from the emitting end of the illumination optical fiber 22. The illumination optical system 29 has a plurality of lenses 33, which are held by a mirror frame 34 such that the optical axes of the lenses 33 are aligned with one another. The plurality of lenses 33 thus held are configured to substantially condense laser light emitted from the emitting end of the illumination optical fiber 22 onto the object obj.

The mirror frame 34 has, at the end on the object obj side, a circular opening 35 for allowing white light emitted from the illumination optical fiber 22 to pass therethrough, where an annular part 36 for holding the lenses 33 is formed. The annular part 36 is colored black on the inner surface, and serves as a reducing part which substantially shields incident light to thereby reduce the passage thereof. The annular part 36 is disposed on the object obj side relative to an incident pupil position ep of the illumination optical system 29. The mirror frame 34 is formed such that the center of the opening 35 of the annular part 36 overlaps the optical axis.

In design, the illumination optical fiber 22 is supported within the tip end part 27 via the angular tube 32 and an attachment ring 37 such that the optical axis of the illumination optical fiber 22 when not oscillated is aligned with the optical axis of the illumination optical system 29. Accordingly, the annular part 36 serving as a reducing part has an inner side surface disposed at a predetermined position relative to the designed optical axis of the illumination optical fiber 22 when not oscillated. Specifically, the inner side surface of the annular part 36 in Embodiment 1 is disposed at a position where the center of the opening 35 overlaps the optical axis.

The detection lens is disposed to take thereinto, as signal light, light resulting from laser light condensed onto the object obj to be reflected, scattered, and refracted by the object obj (light interacted with the object obj), and fluorescence or the like, and then condenses and couples the light to the detection optical fiber bundle 26 disposed behind the detection lens.

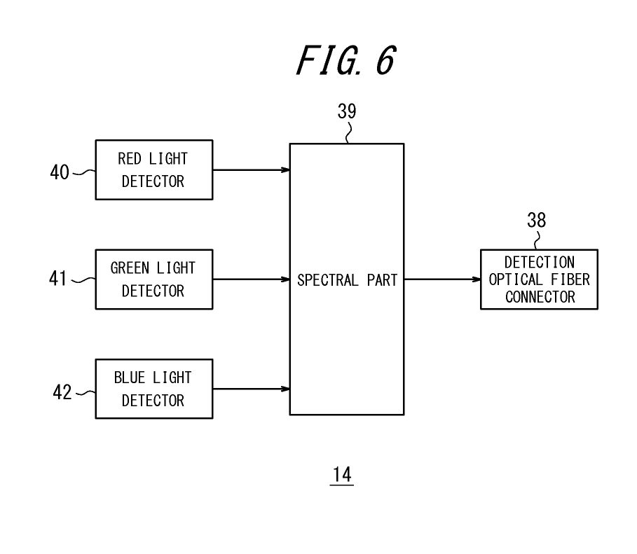

As illustrated in FIG. 6, the detection part 14 is configured by including: a detection optical fiber connector 38, a spectral part 39; a red light detector 40; a green light detector 41; and a blue light detector 42. The detection optical fiber connector 38 optically connects to the detection optical fiber bundle 26, so as to obtain signal light from the detection optical fiber bundle 26. The spectral part 39 is composed of, for example, a dichroic mirror and cross dichroic prism, and separates signal light into red light, green light, and blue light. The red light detector 40 is, for example, a photomultiplier tube or a photodiode, and detects the received amount of red light separated by the spectral part 39. The green light detector 41 is, for example, a photomultiplier tube or a photodiode, and detects the received amount of green light separated by the spectral part 39. The blue light detector 42 is, for example, a photomultiplier tube or a photodiode, and detects the received amount of blue light separated by the spectral part 39.

The controller 15 (see FIG. 1) controls the respective components of the optical scanning observation apparatus 10. The optical scanning observation apparatus 10 has, as its exemplary operating modes, an eccentricity calculation mode and an observation mode, and the controller 15 controls each component according to the eccentricity calculation mode and the observation mode. In below, description is given of control to be carried out by the controller 15 in each mode.

In the eccentricity calculation mode, the optical scanning observation apparatus 10 calculates the eccentricity between the designed position of the optical axis of the optical fiber 22 when not oscillated and the actual position of the optical axis of the actually-attached illumination optical fiber 22.

In the eccentricity calculation mode, the controller 15 causes each of the red light source 17, the green light source 18, and the blue light source 19 of the light source part 11 to radiate a continuous wave, so as to cause the light source part 11 to emit white light.

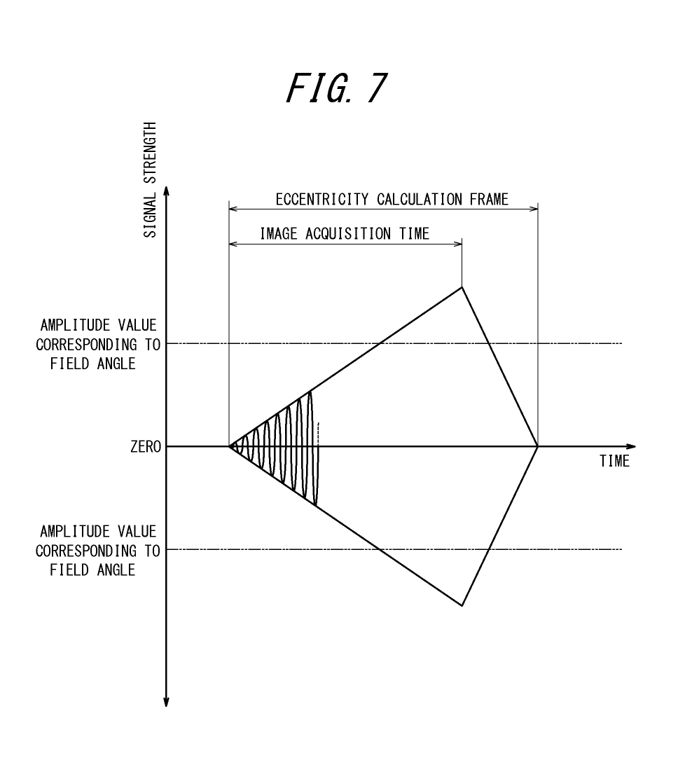

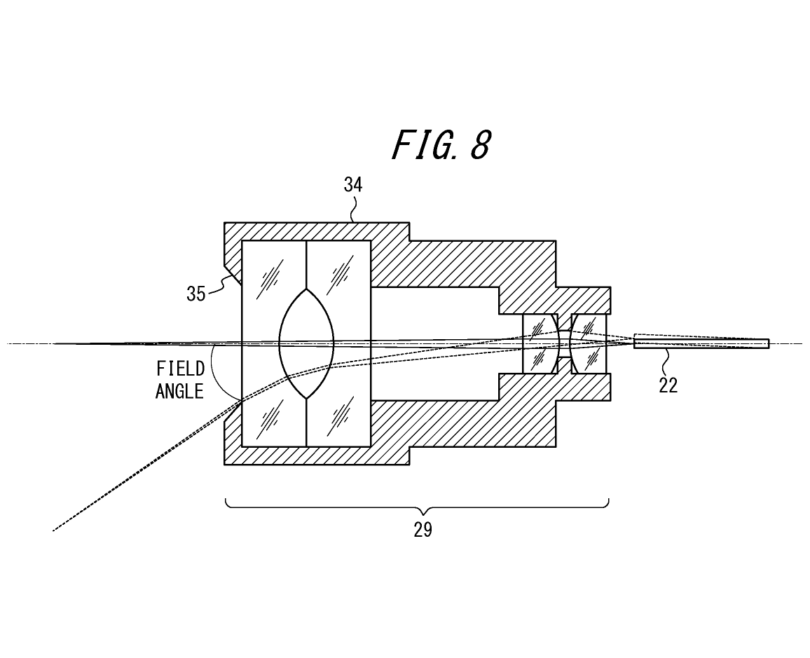

In the eccentricity calculation mode, the controller 15 causes the drive current generator 12 to generate an eccentricity calculation drive signal, the drive signal having a waveform shown in FIG. 7. The eccentricity calculation drive signal vibrates the emitting end so as to deflect the emitting end at a deflection angle larger than the angle of the emitting end of the illumination optical fiber 22, where the deflection angle corresponds to the field angle (see FIG. 8) defined by the illumination optical system 29 and the opening 35 of the mirror frame 34. Accordingly, white light does not always pass through the opening 35 through the entire frame period assigned for eccentricity calculation, and may be shielded by the annular part 36.

In the eccentricity calculation mode, the controller 15 (see FIG. 1) acquires, from the detection part 14, the received amounts of red light, green light, and blue light. Further, the controller 15 estimates, based on the drive signal acquired from the drive current generator 12, the position of the emitting end of the illumination optical fiber 22 when the received amount of light is acquired. The controller 15 forms, based on multiple combinations of the received amount of light and the position in an eccentricity calculation frame, an eccentricity calculation image 43 (see FIG. 9). The eccentricity calculation image 43 includes a light shielding region 44 which is captured when the white light is emitted to the annular part 36, that is, the white light is shielded; and an effective region 45 which includes an image captured when the white light has passed through the opening 35 to be irradiated onto the object obj. The effective region 45 is positioned within the light shielding region 44 correspondingly to the opening 35 and is circular in shape.

In the eccentricity calculation mode, the controller 15 calculates the gravity center position cg of the effective region 45, in the eccentricity calculation image 43. To calculate the gravity center position cg of the effective region 45, the trajectory of the boundary between the light shielding region 44 and the effective region 45 may be calculated through, for example, binary coded processing, to thereby calculate the gravity center position cg based on the trajectory. Further, the controller 15 calculates, as the eccentricity, the displacement of the gravity center position cg from the center position cp of the eccentricity calculation image 43.

In the observation mode, the optical scanning observation apparatus 10 captures an image of the object obj, and displays the image thus captured on the display part 16 (see FIG. 1).

In the observation mode, the controller 15 causes, as in the case of the eccentricity calculation mode, the red light source 17, the green light source 18, and the blue light source 19 of the light source part 11 to radiate a continuous wave, to thereby cause the light source part 11 to emit white light.

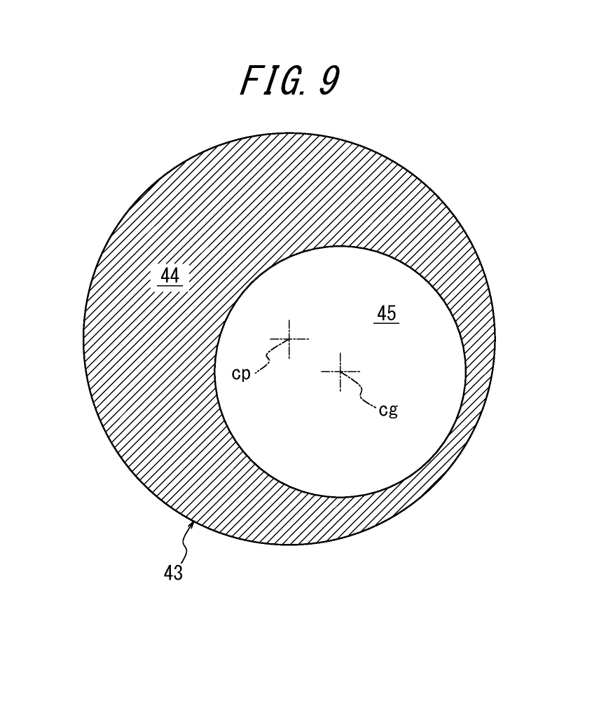

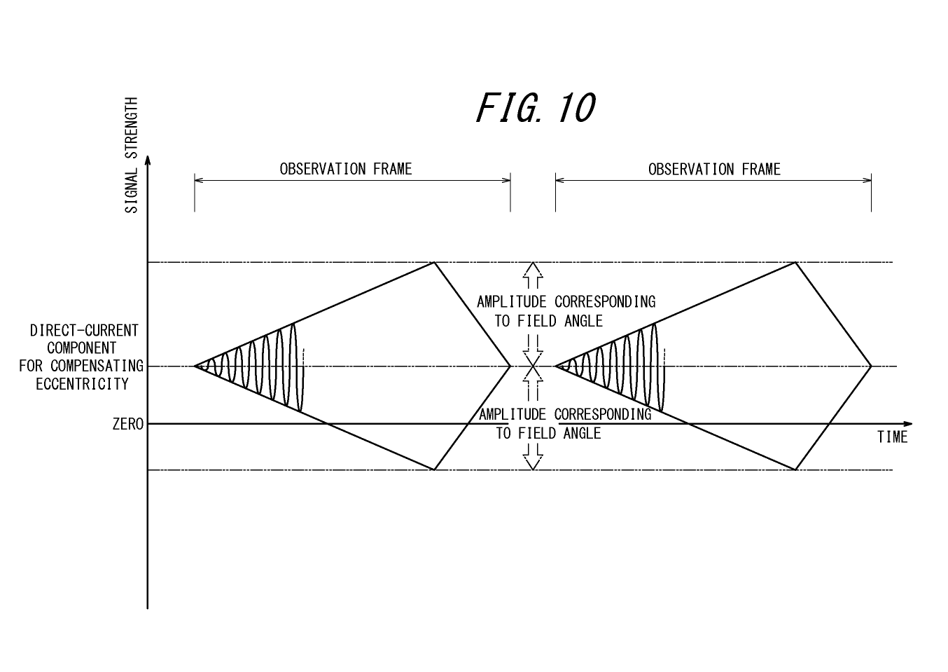

In the observation mode, the controller 15 causes the drive current generator 12 to generate an observation drive signal having a waveform shown in FIG. 10. The observation drive signal vibrates the emitting end so as to deflect the emitting end at a deflection angle smaller than the angle of the emitting end of the illumination optical fiber 22 which corresponds to the field angle (see FIG. 8) defined by the illumination optical system 29 and the opening 35 of the mirror frame 34. Further, the observation drive signal includes a direct-current component for compensating the eccentricity calculated in the eccentricity calculation mode (see FIG. 10). The aforementioned drive signal may be supplied to the actuator 28, to thereby allow the white light emitted from the emitting end to pass through the inside of the opening 35 without being shielded by the annular frame 36, through the entire frame period assigned for observation (main purpose frame period), so as to scan the object obj.

In the observation mode, the controller 15 (see FIG. 1) acquires, from the detection part 14, the received amount of red light, green light, and blue right. Further, the controller 15 estimates, based on the drive signal acquired from the drive current generator 12, the position of the emitting end of the illumination optical fiber 22 when the received amount of light is acquired. The controller 15 forms, based on multiple combinations of the received amount of light and the position in the observation frame, an observation image. Further, the controller 15 causes the display part 16 to display the observation image thus formed.

The controller 15 automatically carries out the eccentricity calculating mode. For example, the controller 15 carries out the eccentricity calculating mode upon detecting an input of switching the operation mode to the observation mode, or before the execution of the observation mode. Alternatively, the controller 15 carries out the eccentricity calculating mode upon detecting an input of switching the operation mode from the observation mode to another mode, between the switching of the modes, or after the completion of the observation mode. Otherwise, the controller 15 carries out the eccentricity calculating mode so as to insert an eccentricity calculation frame in between the observation frames, for example, in 30-minute periods, during the execution of the observation mode.

Next, referring to the flowchart of FIG. 11, description is given of an eccentricity calculating process to be carried out by the controller 15 in the eccentricity calculating mode. As described above, the eccentricity calculating process is started when the operation mode is switched to the eccentricity calculating mode under predetermined conditions.

In Step S100, the controller 15 causes the light source part 11 to radiate white light, so as to emit white light from the emitting end of the illumination optical fiber 22. When the emission of white light is started, the process proceeds to Step S101.

In Step S101, the controller 15 causes the drive current generator 12 to generate a drive signal for eccentricity calculation and to start the transmission of the resulting signal to the actuator 28. When the transmission of the drive signal for eccentricity calculation is started, the process proceeds to Step S102.

In Step S102, the controller 15 acquires the received amount of red light, green light, and blue light detected by the detection part 14. When the received amount of light is acquired, the process proceeds to Step S103.

In Step S103, the controller 15 estimates, based on a drive signal acquired from the drive current generator 12, the displaced position of the emitting end when the received amount of light is detected in Step S102. When the displaced position is estimated, the process proceeds to Step S104.

In Step S104, the controller 15 identifies whether or not it is in the image acquisition period (see FIG. 7) in the eccentricity calculation frame. When it is in the image acquisition period, the process returns to Step S102 (see FIG. 11). When the image acquisition is completed, the process proceeds to Step S105.

In Step S105, the controller 15 forms an image 43 for eccentricity calculation using a plurality of combinations of the received amount of light and the estimated position acquired during the image acquisition period. When the eccentricity calculation image 43 is formed, the process proceeds to Step S106.

In Step S106, the controller 15 calculates the gravity center position cg of the effective region 45 in the eccentricity calculation image 43 formed in Step S105. When the gravity center position cg is calculated, the process proceeds to Step S107.

In Step S107, the controller 15 calculates eccentricity, based on the gravity center position cg calculated in Step S106. The controller 15 stores the eccentricity thus calculated in a memory owned by the controller 15. When the eccentricity is calculated, the eccentricity calculation process is ended.

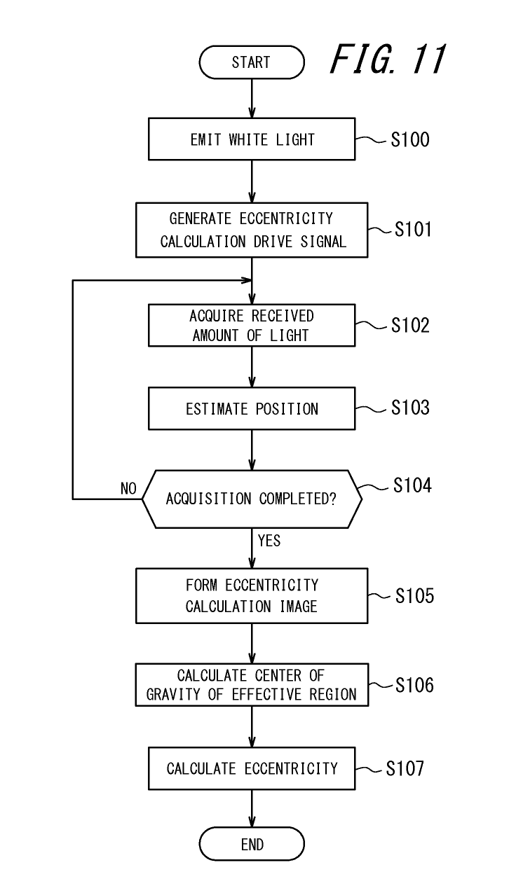

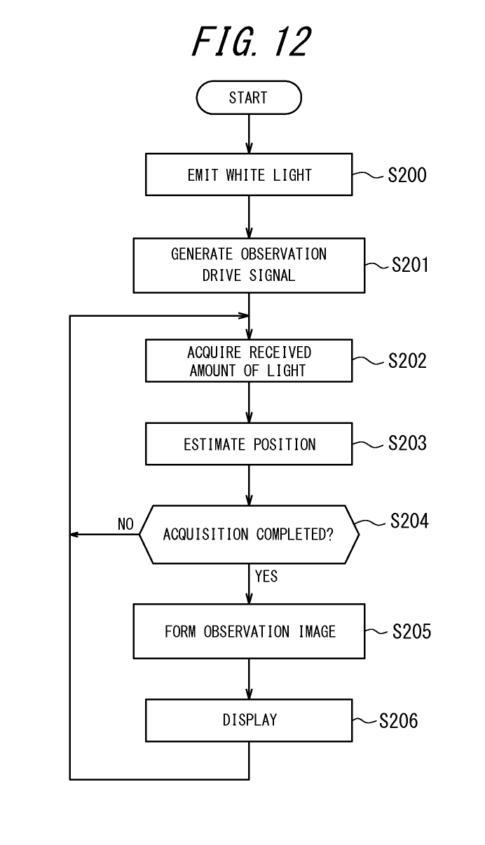

Next, referring to the flowchart of FIG. 12, description is given of an observation process to be carried out by the controller 15 in the observation mode. As described above, the observation process is started after the detection of an input to switch the operation mode to the observation mode. Further, the observation process is ended after the detection of an input to switch the operation mode from the observation mode to another mode.

In Step S200, the controller 15 causes the light source part 11 to radiate white light, so as to emit white light from the emitting end of the illumination optical fiber 22. When the emission of white light is started, the process proceeds to Step S201.

In Step S201, the controller 15 causes, based on the eccentricity stored in the memory, the drive current generator 12 to generate an observation drive signal and to start transmitting the drive signal to the actuator 28. When the transmission of the observation drive signal is started, the process proceeds to Step S202.

From Steps S202 to S204, the controller 15 carries out the same operation as in from Steps S102 to S104 in the eccentricity calculation process. When the image acquisition is completed in Step S204, the process proceeds to Step S205.

In Step S205, the controller 15 uses a plurality of combinations of the received amount of light acquired in the image acquisition period and the estimated position to form an observation image. When the observation image is formed, the process proceeds to Step S206.

In Step S206, the controller 15 transmits the observation image formed in Step S205 to the display part 16 to cause the image to be displayed thereon. When the observation image is displayed, the process returns to Step S102.

According to the optical scanning unit of Embodiment 1 configured as described above, an image is captured in a state where the reducing part disposed at a predetermined position with respect to the designed optical axis of the illumination optical fiber 22 is caused to irradiate light, and thus, the eccentricity can be calculated from the captured image. With this configuration, the eccentricity can be calculated with a simple structure, and based on the eccentricity, the eccentricity of the illumination optical fiber 22 can be compensated and the image can be corrected.

Further, according to the optical scanning unit of Embodiment 1, the eccentricity is calculated using only red light, green light, and blue light that are minimally necessary for forming a color image in the observation mode, and thus, there is no need complicate the structure as compared with a conventional optical scanning unit.

According to the optical scanning unit of Embodiment 1, the eccentricity is calculated in the eccentricity calculation frame different from the observation frame, and thus, even if the scan carried out in the eccentricity calculation frame is inappropriate for forming an observation image, an image to be displayed on the display part 16 should suffer reduced effect.

According to the optical scanning unit of Embodiment 1, the annular part 36 serving as a reducing part is in a shape that surrounds around the opening 35, and thus boundary between the light shielding region 44 and the effective region 45 in the eccentricity calculation image 43 can easily be detected and the gravity center position cg of the image can also be easily detected.

In the optical scanning unit of Embodiment 1, part of the mirror frame 34 is caused to serve as the reducing part, which makes it possible to simplify the configuration as compared with a configuration incorporating the reducing part incorporated as a separate member.

Further, according to the optical scanning unit of Embodiment 1, the illumination optical fiber 22 is vibrated so as to be deflected at an angle larger than the inclination angle of the emitting end, corresponding to a predetermined field angle, when in the eccentricity calculation frame; and the illumination optical fiber 22 is vibrated so as to be deflected at the inclination angle of the emitting end, corresponding to a predetermined field angle, when in the observation frame. Thus, in the observation mode, wasteful emission of white light and wasteful detection of signal light can be suppressed.

### Embodiment 2

Next, Embodiment 2 of the present disclosure is described. Embodiment 2 is different from Embodiment 1 in configuration of the light source part, the reducing part, and the detection part. In the following, description is given of Embodiment 2, focusing the differences from Embodiment 1. Here, like parts as those of Embodiment 1 are denoted by like reference symbols.

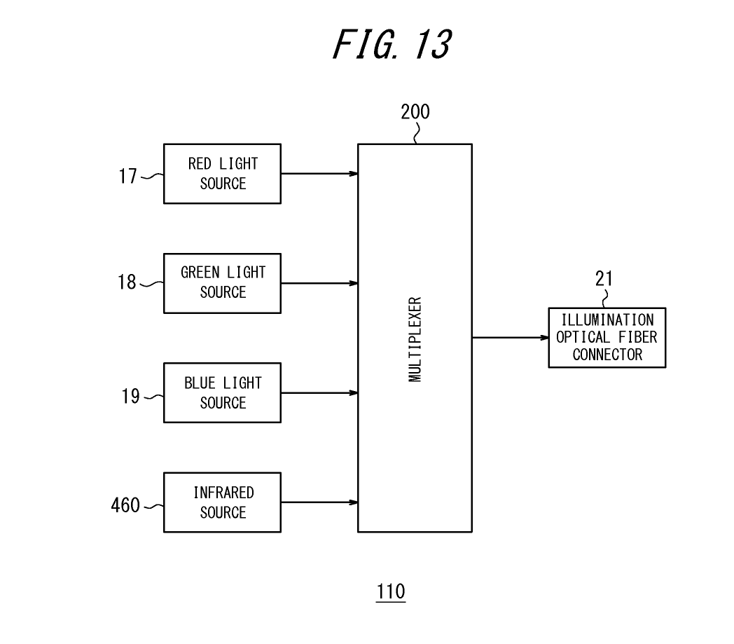

A light source part 110 is configured by including, as illustrated in FIG. 13: the red light source 17; the green light source 18; the blue light source 19; an infrared source 460; a multiplexer 200; and the illumination optical fiber connector 21. The red light source 17, the green light source 18, the blue light source 19, and the illumination optical fiber connector 21 are similar in configuration and function to those of Embodiment 1. The infrared source 460 may be, for example, an infrared laser, and radiates infrared light of 800 nm in wavelength. As in Embodiment 1, the multiplexer 200 may include, for example, a dichroic mirror and a fiber combiner, and multiplexes red light, green light, blue light, and infrared light each radiated by the red light source 17, the green light source 18, the blue light source 19, and the infrared source 460, respectively.

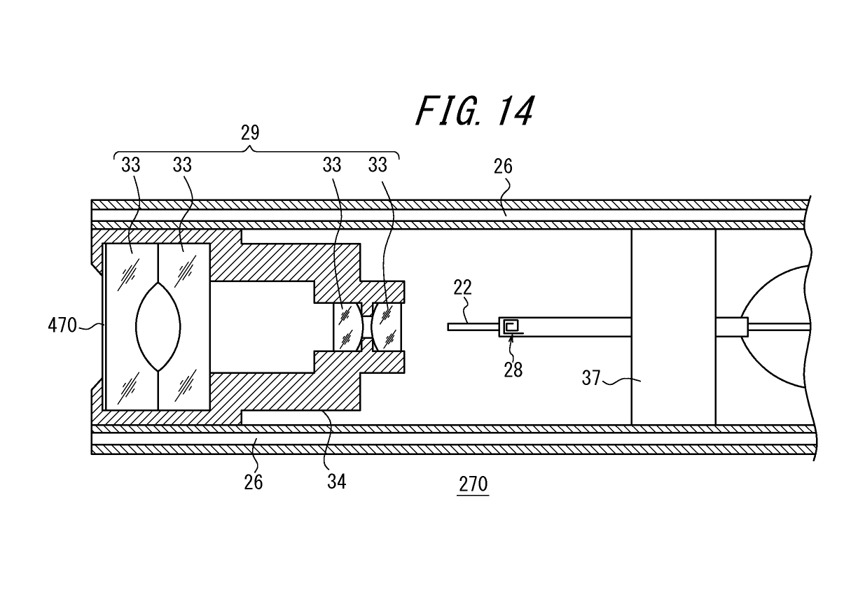

As illustrated in FIG. 14, the tip end part 270 of the optical scanning endoscope main body 13 has the actuator 28, the illumination optical system 29, the detection lens, and a plate 470, with the illumination optical fiber 22 and the detection optical fiber bundle 26 extending therethrough. The actuator 28, the illumination optical system 29, the detection lens, the illumination optical fiber 22, and the detection optical fiber bundle 26 are similar to those of Embodiment 1 in configuration, function, and arrangement.

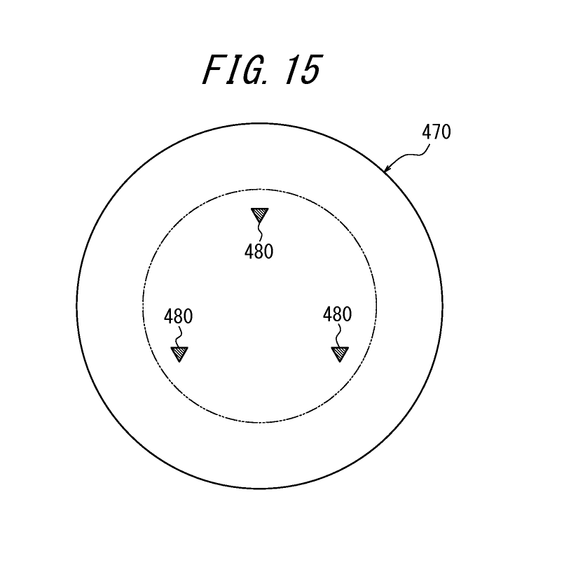

As illustrated in FIG. 15, the plate 470 is formed of a transparent circular disk which transmits visible light, and has a light shielding part 480 disposed thereon for transmitting visible light while shielding infrared light. The light shielding part 480 is formed by applying a coating which transmits visible light and shields infrared light. The light shielding part 480 is formed within a region (see the dashed line) corresponding to the inside of the opening 35, with the plate 470 being attached to the mirror frame 34. The light shielding parts 480 are formed, for example, at three different positions such that the gravity center of all the light shielding parts 480 coincides with the center of the plate 470. The plate 470 is attached to the mirror frame 34 so as to be disposed on the object obj side relative to the illumination optical system 29 in such a manner that the center thereof coincides with the optical axis of the illumination optical system 29 (see FIG. 14).

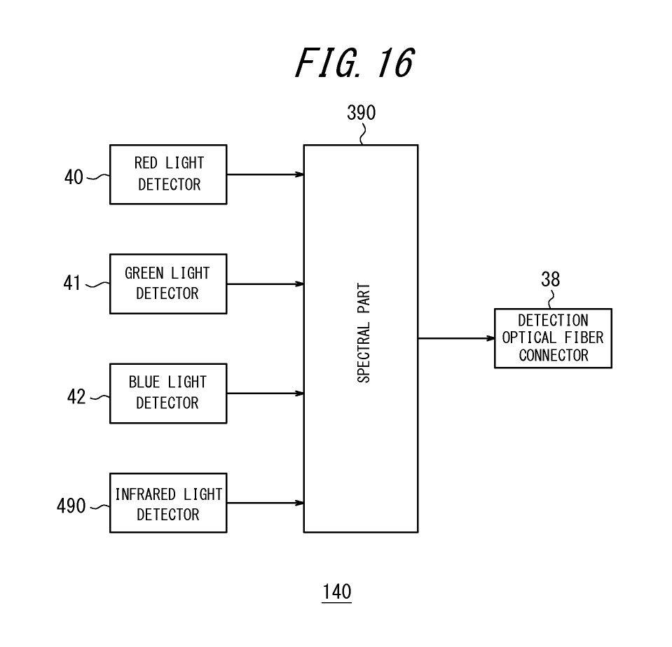

As illustrated in FIG. 16, a detection part 140 is configured by including: a detection optical fiber connector 38, a spectral part 390; the red light detector 40; the green light detector 41; the blue light detector 42; and an infrared light detector 490. The detection optical fiber connector 38, the red light detector 40, the green light detector 41, and the blue light detector 41 are similar in configuration and function to those of Embodiment 1. The spectral part 390 is composed of, as in Embodiment 1, for example, a dichroic mirror and a cross dichroic prism, and separates signal light into red light, green light, blue light, and infrared light. The infrared light detector 490 is, for example, a photomultiplier tube or a photodiode, and detects the received amount of infrared light separated by the spectral part 390.

The controller 15 controls, as in Embodiment 1, the respective components of the optical scanning observation apparatus 10. In Embodiment 2, the optical scanning observation apparatus 10 does not have the eccentricity calculating mode, and has an observation mode different from that of Embodiment 1. In below, description is given of control to be carried out by the controller 15 in the observation mode of Embodiment 2.

In the observation mode, the controller 15 causes the red light source 17, the green light source 18, and the blue light source 19 of the light source part 11 to radiate a continuous wave, so as to cause the light source part 11 to emit white light. Further, the controller 15 causes the infrared source 460 to radiate a continuous wave only in one frame period at predetermined timing in the observation mode, such as at the start of the observation mode, at timing in a predetermined period during the execution of the observation mode, and at the end of the observation mode.

In the observation mode, the controller 15 causes the drive current generator 12 to generate the observation drive signal of Embodiment 1. However, when causing the light source part 11 to emit infrared light, the controller 15 causes the drive current generator 12 to generate a drive signal with zero eccentricity, i.e., a drive signal having no direct-current component.

In the observation mode, the controller 15 acquires, from the detection part 140, the received amount of red light, green light, and blue light. The controller 15 further estimates, based on the drive signal to be acquired from the drive current generator 12, the position of the emitting end of the illumination optical fiber 22 at the acquisition of the received amount of light. The controller 15 forms an observation image, based on multiple combinations of the received amount of light and the position in a single frame. The controller 15 also causes the display part 16 to display the observation image thus formed.

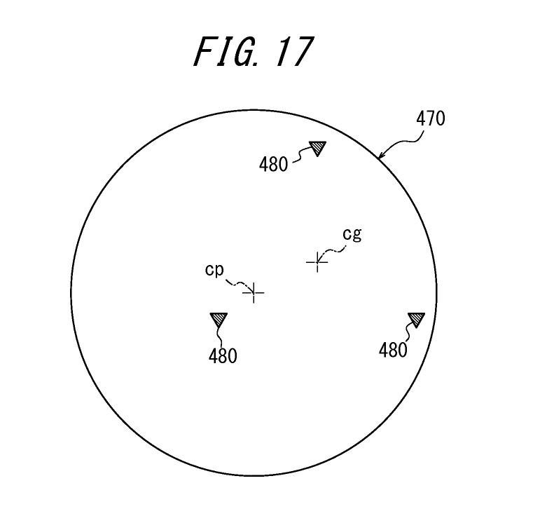

However, when causing the light source part 11 to emit infrared light, the controller 15 also acquires, from the detection part 140, the received amount of infrared light. The controller 15 further estimates, based on a drive signal to be acquired from the drive current generator 12, the position of the emitting end of the illumination optical fiber 22 at the acquisition of the received amount of infrared light. Further, the controller 15 forms an eccentricity calculation image 430 shown in FIG. 17, based on multiple combinations of the received amount of infrared light and the position in a single frame. Further, the controller 15 detects the positions of the light shielding parts 480 in the eccentricity calculation image 430 so as to calculate the gravity center position cg of all the light shielding parts 480. Further, the controller 15 calculates, as the eccentricity, the displacement of the gravity center position cg from the center position cp of the eccentricity calculation image 430. The eccentricity thus calculated is used for generating an observation drive signal.

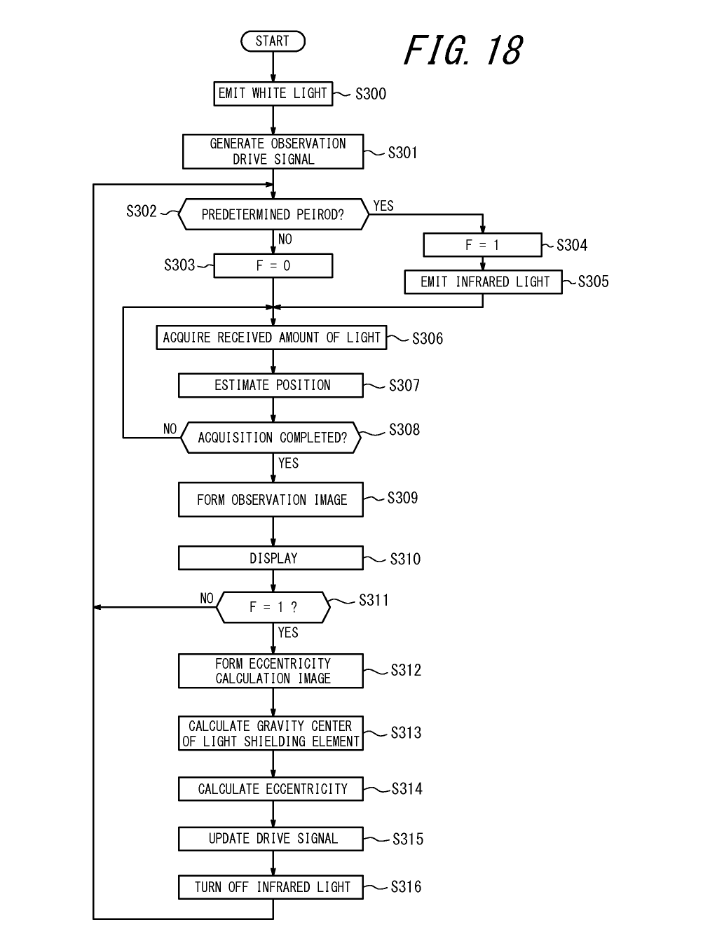

Next, with reference to the flowchart of FIG. 18, description is given of an observation process to be carried out by the controller 15 in the observation mode. The observation process is started after the detection of an input to switch the operation mode of the optical scanning observation apparatus 10 to the observation mode. Then, the observation process is ended after the detection of an input to switch the operation mode from the observation mode to another mode.

In Steps S300 and S301, the controller 15 carries out the same operation as in Steps S200 and S201 in the observation process of Embodiment 1. When the observation drive signal is generated, the process proceeds to Step S302.

In Step S302, the controller 15 judges whether the current point in time falls within a predetermined period assigned for carrying out eccentricity calculation. When the current point in time does not fall within the predetermined period, the process proceeds to Step S303. When the current point in time falls within the predetermined period, the process proceeds to Step S304.

In Step S303, the controller 15 sets 0 to a calculation period judgment flag F. When the setting of the flag F is completed, the process proceeds to Step S306.

In Step S304, the controller 15 sets 1 to the calculation period judgment flag F. When the setting of the flag F is completed, the process proceeds to Step S305.

In Step S305, the controller 15 causes the light source part 11 to radiate infrared light along with white light, so as to emit white light including infrared light from the emitting end of the illumination optical fiber 22. When the emission of infrared light is started, the process proceeds to Step S306.

From Steps S306 to S310, the controller 15 carries out the same operation as in from Steps S202 to S206 in the observation process of Embodiment 1. When the observation image is displayed on the display part 16, the process proceeds to Step S311.

In Step S311, the controller 15 judges whether the calculation period judgment flag F is 1. When the calculation period judgment flag F is 1, the process proceeds to Step S312. When the calculation period judgment flag F is 0, the process returns to Step S302.

In Step S312, the controller 15 forms the eccentricity calculation image 430 using multiple combinations of the received amount of infrared light and the estimated position which have been acquired during the image acquisition period. When the eccentricity calculation image 430 is formed, the process proceeds to Step S313.

In Step S313, the controller 15 calculates the gravity center position cg of the light shielding parts 480 on the eccentricity calculation image 430 formed in Step S312. When the gravity center position cg is calculated, the process proceeds to Step S314.

In Step S314, the controller 15 calculates the eccentricity, based on the gravity center position cg calculated in Step S313. When the eccentricity is calculated, the process proceeds to Step S315.

In Step S315, the controller 15 calculates, based on the eccentricity calculated in Step S314, a direct-current component for compensating the eccentricity, and uses the direct-current component thus calculated to update the drive signal. The controller 15 uses the drive signal thus updated, so as to drive the actuator 28 in the following stages. When the drive signal is updated, the process proceeds to Step S316.

In Step S316, the controller 15 causes the light source part 11 to turn off infrared light, so as to switch the emission thereof to white light emission only. When infrared light is turned off, the process proceeds to Step S302.

The optical scanning unit of Embodiment 2 configured as described above is also capable of calculating the eccentricity from a captured image, as in Embodiment 1. Accordingly, the eccentricity can be calculated by a simple configuration, allowing for compensation of the eccentricity of the illumination optical fiber 22 or correction of the image.

Further, according to the optical scanning unit of Embodiment 2, the eccentricity is calculated using infrared light of a different bandwidth from light of three colors (red light, green light, and blue light) for forming a color image, which means the eccentricity can be calculated without stopping the formation of an observation image. The eccentricity can be calculated using infrared light that does not contribute to the formation of a color image, and thus, there is no need to change the maximum amplitude of the emitting end in order for calculating the eccentricity.

The present disclosure has been described with reference to the accompanying drawings and Embodiments, but it should be noted that various modifications and alterations based on the present disclosure can readily be available to a person skilled in the art. Therefore, it should also be noted that such modifications and alterations are all fall within the scope of the present disclosure.

For example, in Embodiment 1 and Embodiment 2, the annular part 36 and the light shielding part 480 each serving as the reducing part are configured to shield specific light, which however may also be configured to reduce the transmission amount. Even if configured to reduce the transmission amount, a region where the received amount of light is relatively low may still be identified through, for example, binarization so as to be nevertheless identifiable as a region of the reducing part.

Further, the actuator 28 of Embodiment 1 and Embodiment 2 is an electromagnetic actuator, but not limited to an electromagnetic actuator. For example, the actuator 28 may be a piezoelectric actuator. When the actuator 28 is a piezoelectric actuator, an alternating voltage may be applied as the drive signal.

Further, in Embodiment 1 and Embodiment 2, the optical scanning observation apparatus 10 is an optical scanning endoscope apparatus, but may be other observation apparatuses. For example, the optical scanning observation apparatus 10 may be an optical scanning microscope apparatus.

Further, Embodiment 1 and Embodiment 2 are configured to generate a drive signal for displacing the emitting end of the illumination optical fiber 22 in a swirling manner so as to spirally scan the object, but the way of scanning is not limited to spiral scan. For example, the emitting end of the illumination optical fiber 22 may be driven so as to raster scan or Lissajous scan the object.

Further, in Embodiment 1 and Embodiment 2, the light source part 11 is configured to radiate a plurality of continuous optical waves of different bandwidths. However, the light source part 11 may be configured to repeatedly radiate lights of respective bandwidths in order from each light source of the light source part 11, as in the dot sequential system. In the dot sequential system as described above, the detection parts 14, 140 may use a single detector for detecting the received amount of lights of respective bandwidths.

Further, in Embodiment 1, the annular part 36 is configured to shield white light. However, the annular part 36 may also be configured to shield light having part of the bandwidth included in white light.

Further, in Embodiment 1, the annular part 36 in a shape surrounding the opening 35 is configured to serve as a reducing part. However, as in Embodiment 2, a region for reducing the transmission of white light may be disposed in a region corresponding to the opening 35.

Further, in Embodiment 1, the opening 35 of the mirror frame 34 is circular in shape. However, the opening 35 may be formed in a rectangular shape when scanning a rectangular region of the object obj as in the case of raster scan or Lissajous scan.

Further, in Embodiment 1, the inner surface of the annular part 36 is colored black to serve as the reducing part. However, an optical element constituting the illumination optical system 29, such as, for example, the lens 33 on the most object obj side may be applied, on the surface thereof, with a coating form a film thereon, to thereby serve as the reducing part.

Further, Embodiment 2 is configured to detect the position of the light shielding part 480 using infrared light. However, there may be used light of other bandwidths not used for forming the observation image. For example, there may be used light in an ultraviolet region or light in a visible bandwidth other than the light of three colors (red light, green light, and blue light) to be used for forming the observation image as a color image.

Further, in Embodiment 2, the light shielding part 480 is provided in a region corresponding to the opening 35 of the mirror frame 34. However, as in Embodiment 1, the light shielding part 480 may be formed in a shape of surrounding the opening 35, as in Embodiment 1. The mirror frame 34 may be formed of a transparent material at the end on the object obj side, making it possible to improve the detection accuracy of the contour of the opening 35 while increasing the field angle, which means that the calculation accuracy of the eccentricity can be improved.

### Embodiment 3

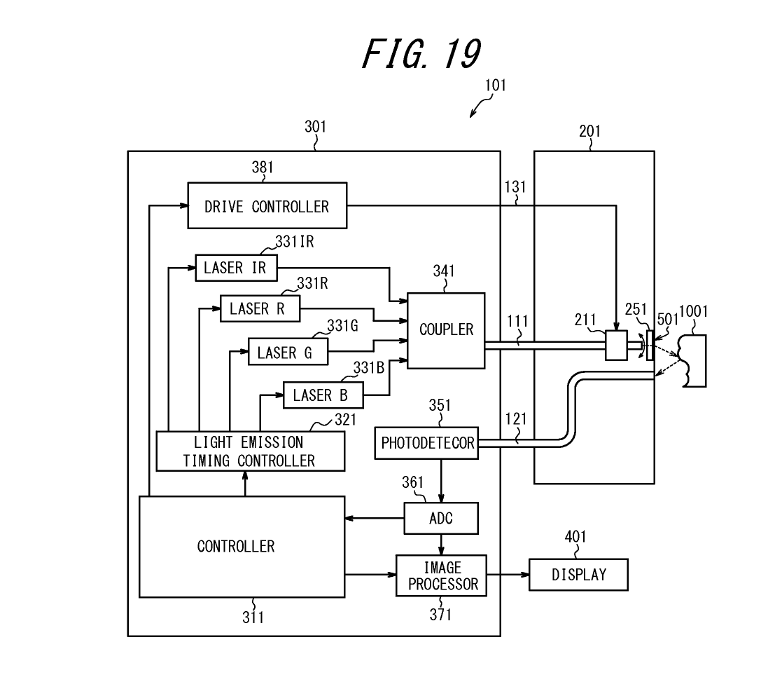

FIG. 19 is a block diagram illustrating a schematic configuration of a fiber scanning endoscope apparatus as an example of the optical fiber scanning apparatus according to Embodiment 3. The fiber scanning endoscope apparatus 101 includes: a scope 201; a control main body 301; and a display 401.

The control main body 301 is configured by including: a controller 311 for controlling the whole of the fiber scanning endoscope apparatus 101; a light emission timing controller 321; lasers 331R, 331G, 331B, 331IR, and a coupler 341. The light emission timing controller 321 controls, under the control of the controller 311, the light emission timings of three lasers 331R, 331G, 331B for emitting laser light of three primary colors of red, green, and blue, and the laser 331IR for emitting near-infrared light. The lasers 331R, 331G, 331B, 3311R may employ, for example, a diode pumped solid state (DPSS) laser and a laser diode. Laser lights emitted from the lasers 331R, 331G, 331B, 331IR are multiplexed by the coupler 341, and caused to be incident on an illumination optical fiber 111 as a single mode fiber. Needless to say, the configuration of the light source part of the fiber scanning endoscope apparatus 101 is not limited thereto. For example, the light source part may employ one visible light laser source and one near-infrared light source, or may employ a plurality of other light sources. Alternatively, in place of the laser for emitting near-infrared light, there may be employed a laser that emits light having any other wavelength that does not contribute to image formation. In particular, it is preferred to select light having a wavelength that does not coincide with the absorption wavelength of an object 1001. Further, the lasers 331R, 331G, 331B, 331IR, and the coupler 341 may be accommodated in a casing separate from the control main body 301, the casing being connected to the control main body 301 via a signal line.

The illumination optical fiber 111 is linked to the tip end part of the scope 201, and light incident from the coupler 341 to the illumination optical fiber 111 is guided to the tip end part of the scope so as to be irradiated toward the object 1001. At this time, an actuator 211 is vibratory driven, to thereby allow the illumination light emitted from the illumination optical fiber 111 to two-dimensionally scan the observation surface of the object 1001. The actuator 211 is controlled by a drive controller 381 to be described later of the control main body 301. Signal light (light to be detected) including reflected light, scattered light, and fluorescence obtained from the object 1001 irradiated with illumination light is received by each of the tip ends of detection optical fibers 121 formed of a plurality of multi mode fibers, so as to travel through within the scope 201 so as to be guided to the control main body 301.

The control main body 301 further includes: a photodetector 351 (detection part) for processing signal light; an analog-digital convertor (ADC) 361; and an image processor 371. The photodetector 351 splits the signal light having travelled through the detection optical fibers 121 into spectral components corresponding to the respective wavelengths of the lasers 331R, 331G, 331B, 331IR, and converts each of the spectral components into an electric signal by a photodiode or the like. The ADC 361 converts, into a digital signal, the signal of the signal light having been converted into the electric signal, and outputs the signal of near-infrared light to the controller 311 while outputting signals of other spectral components to the image processor 371 (image acquisition part). The controller 311 extracts, along with time, variation in signal strength of the near-infrared light, and compensates the scanning orbit of the emitting end 111c of the illumination optical fiber 111 as will be described later. Further, the controller 311 calculates information on the scanning position on the scanning route, based on such information as the scan start time, and the amplitude and phase of a vibration voltage applied by the drive controller 381, or extracts information on the scanning position from a previously-prepared table in which the scanning position information is associated with the time elapsed from the scanning start, and passes the information to the image processor 371. The image processor 371 acquires, from the digital signal output from the ADC 36, pixel data on the object 1001 at the relevant scanning position. The image processor 371 sequentially stores information on the scanning position and the pixel data in a memory (not shown), subjects the information to necessary processing such as interpolation processing after the completion of scan or during the scan so as to generate an image of the object 1001, and displays the image on the display 401.

In each process above, the controller 311 synchronously controls the light emission timing controller 321, the photodetector 351, the drive controller 381, and the image processor 371.

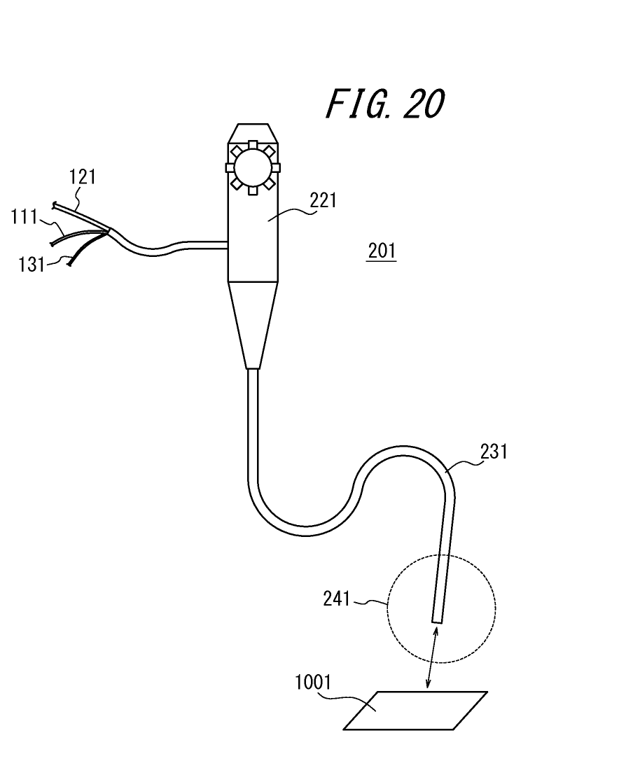

FIG. 20 is a schematic external view of the scope 201. The scope 201 includes an operation portion 221 and an insertion portion 231. The illumination optical fiber 111, the detection optical fibers 121, and a wiring cable 131 from the control main body 301 are connected to the operation portion 221. The illumination optical fiber 111, the detection optical fibers 121, and the wiring cable 131 all pass through inside the insertion portion 231, and are guided to a tip end part 241 (circled by the broken line of FIG. 201) of the insertion portion 231.

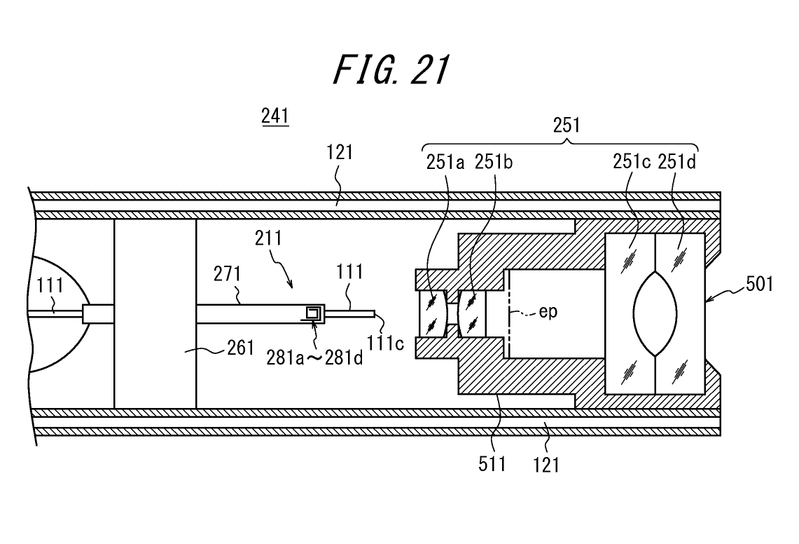

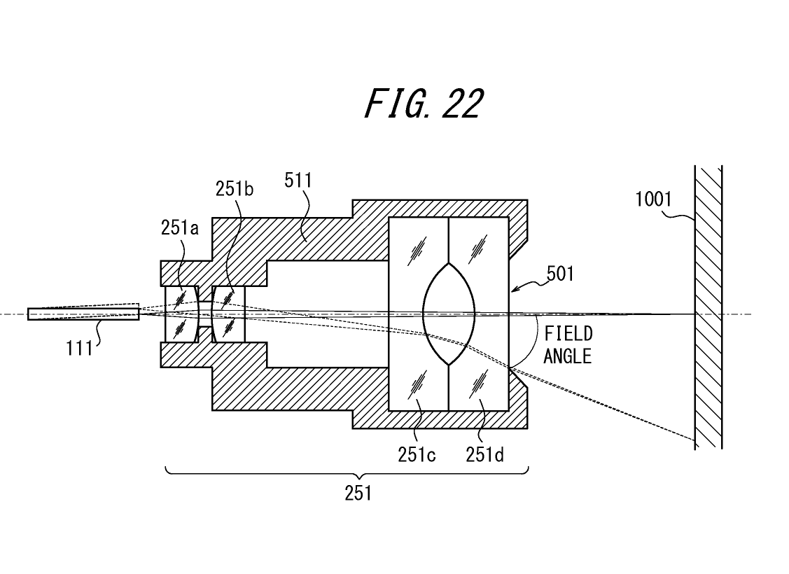

FIG. 21 is an enlarged sectional view of the tip end part 241 of the insertion portion 231 of the scope 201 of FIG. 20. FIG. 22 illustrates the optical system of FIG. 21 and exemplary optical paths of illumination light. The tip end part 241 includes the actuator 211 and a projection optical system 251, in which the illumination optical fiber 111 passes through the central part and a plurality of the detection optical fibers 121 pass through the outer circumferential part. As illustrated in FIGS. 21, 22, the optical system 251 is configured by including a pair of plano-convex lenses 251a, 251b, and a pair of plano-concave lenses 251c, 251d, which are fixed within a mirror frame 511. In either pair of the lenses, two lenses are arranged in such a manner that the curved surfaces are opposed to each other. The lenses 251a to 251d are configured such that laser light emitted from the emitting end of the illumination optical fiber 111 is substantially condensed onto the object 1001. Further, the optical system 251 is formed such that the light flux emitted from the illumination optical fiber 111 in different directions is once converged at a pupil position ep between the plano-convex lenses 251a, 251b and the plano-concave lenses 251c, 251d. It should be noted that the optical system 251 is not limited to the illustrated lens configuration, and may employ various configurations.

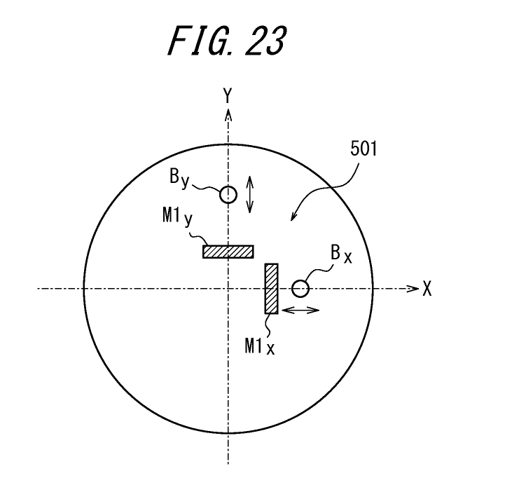

Further, the plano-concave lens 251d farthest to the pupil position ep has a light shielding mask 501 (light reducing part) disposed on the plane on the object 1001 side. FIG. 23 illustrates the light shielding mask 501. The light shielding mask 501 has light shielding regions M1ₓ, M1_{y} each shielding only near-infrared light while transmitting visible light. It should be noted that the light shielding regions M1ₓ, M1_{y} are not required to completely shield near-infrared light, and may transmit part thereof. Here, defined as the origin position is a position where illumination light obtained when the emitting end of the illumination optical fiber 111 is found at the vibration center (or at the rest position) intersects the plane of the plano-concave lens 251d where the light shielding mask 501 is disposed, defined as the X direction is a direction orthogonal to the optical axis, and defined as the Y direction is a direction orthogonal to the X direction and to the optical axis. In this case, the light shielding region M1ₓ is arranged at a position displaced from the origin point in the X direction so as to be formed as a longitudinal rectangular light shielding region elongated in the Y direction. Meanwhile, the light shielding region M1_{y} is arranged at a position displaced from the origin point in the Y direction so as to be formed as a longitudinal rectangular light shielding region elongated in the X direction. In FIG. 23, points Bₓ and B_{y} each represent a scanning point of illumination light that passes through the plane of the plano-concave lens 251d.

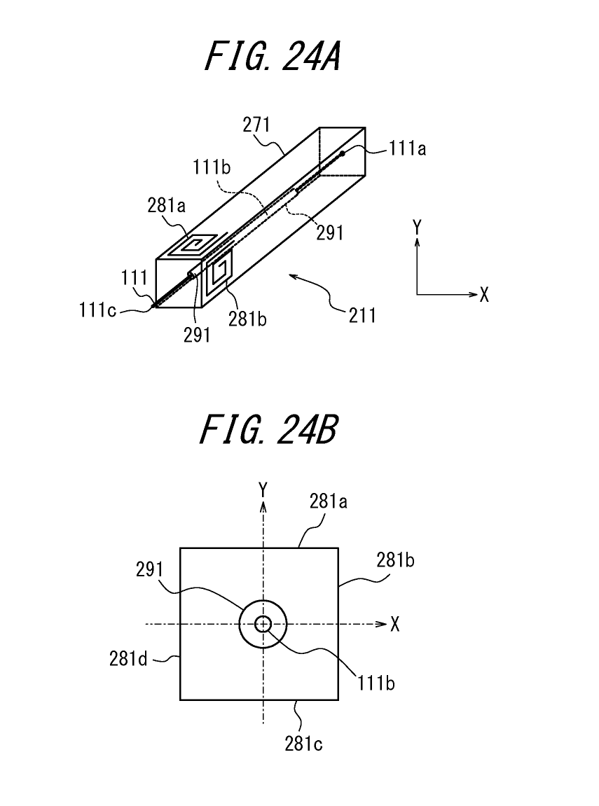

On the other hand, as illustrated in FIG. 21, the actuator 211 is configured by including: an angular tube 271 fixed inside the insertion portion 231 of the scope 201 by means of an attachment ring 261; deflection magnetic field generating coils 281a to 281d arranged on four faces of the rectangular tube 271; and a cylindrical permanent magnet 291 (see FIG. 24) fixed to the outer circumference of the illumination optical fiber 111. The illumination optical fiber 111 has a fixed part 111a cantilevered by the attachment ring 261, and a portion defined between the fixed part 111a and the emitting end 111c is configured to serve as a tip end part 111b supported in an oscillatable manner (see FIG. 24). Meanwhile, the detection optical fibers 121 are arranged so as to pass through the outer circumferential part of the insertion portion 231, and extends to the tip of the tip end part 241 of the scope 201. Further, the emitting end of each of the detection optical fibers 121 is provided with a detection lens (not shown). The detection lens is arranged such that the lens takes thereinto, as signal light, light resulting from the illumination light condensed onto the object 1001 to be reflected, scattered, and reflected by the object 1001, or fluorescence or the like, and then couples the light to each of the detection optical fibers 121.

FIG. 24 each illustrate an actuator positioned at the tip end part of the scope of FIG. 21, in which: FIG. 24A is a perspective view; and FIG. 24B is a sectional view taken along a plane vertical to the axis of the optical fiber of the actuator 211 of FIG. 24A. The actuator 211 includes the deflection magnetic field generating coils 281a to 281d and the permanent magnet 291. Further, the permanent magnet 291, which is magnetized in the axial direction of the illumination optical fiber 111 and in a cylindrical shape having a through hole, is coupled to part of the tip end part 111b of the illumination optical fiber 111 in a state where the illumination optical fiber 111 is passed through the through hole. Further, the rectangular tube 271 fixed at one end to the attachment ring 261 is provided so as to surround the tip end part 111b, and the rectangular tube 271 is provided with the flat-type deflection magnetic field generating coils 281a to 281d which are disposed on the respective side surfaces of the rectangular tube 271 in a section facing one pole of the permanent magnet 291.

The pair of the deflection magnetic field generating coils 281a and 281c in the Y direction and the pair of the deflection magnetic field generating coils 281b and 281d in the X direction are each disposed on the mutually-opposing faces of the angular tube 271, where the line connecting the center of the deflection magnetic field generating coil 281a and the center of the deflection magnetic field generating coil 281c and the line connecting the center of the deflection magnetic field generating coil 281b and the center of the deflection magnetic field generating coil 281d are orthogonal to each other in the vicinity of the central axis of the angular tube 271 along which the illumination optical fiber 111 is disposed when stationary. The coils are connected to the drive controller 381 of the control main body 301 via the wiring cable 131, and driven by a drive current from the drive controller 381.

The drive controller 381 applies a vibration current to the deflection magnetic field generating coils 281b and 281d for X-direction driving, so that the magnetic field is generated always in the same direction. Meanwhile, the drive controller 381 also applies a vibration current to the deflection magnetic field generating coils 281a and 281c for Y-direction driving, so that the magnetic field to be generated is always in the same direction. The vibration current to be applied to the pair of the deflection magnetic field generating coils 281a, 281c may be the same as or different from the vibration current to be applied to the pair of 281b, 281d. The deflection magnetic field generating coils 281a, 281c for Y-direction driving and the deflection magnetic field generating coils 281b, 281d for X-direction driving may each be vibratory driven, which causes to vibrate the tip end part 111b of the illumination optical fiber 111 of FIGS. 21, 24 so as to deflect the emitting end 111c, with the result that the laser light emitted from the emitting end 111c sequentially scans the surface of the object 1001.

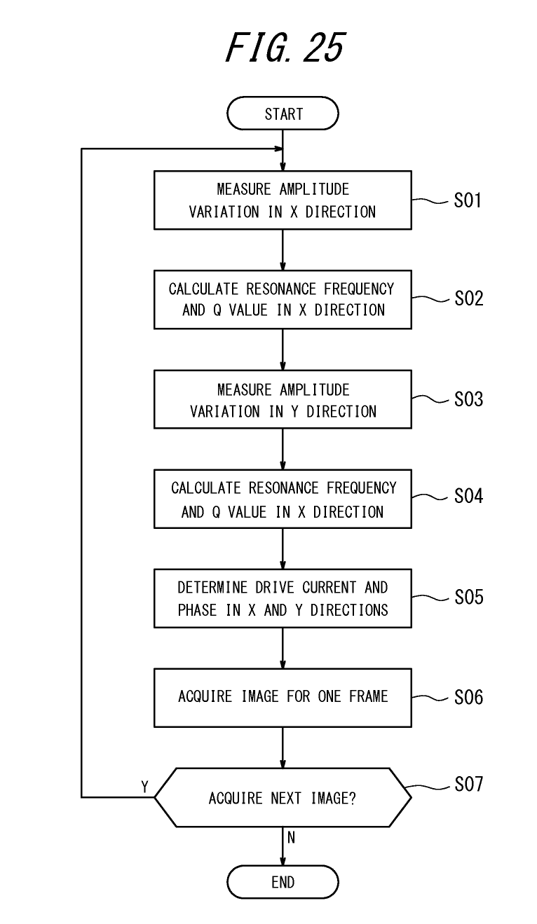

Next, referring to the flowchart of FIG. 25, description is given of an operation of the fiber scanning endoscope apparatus 101. The fiber scanning endoscope apparatus 101 can be operated in two different modes including: a vibration adjustment mode (Steps S01 to S05) for compensating the vibration trajectory of the emitting end 111c of the illumination optical fiber 111; and an image acquisition mode (Step S06) for acquiring image data on the object. The vibration adjustment mode is carried out for each image acquisition for one frame in the image acquisition mode, before and after the image for one frame is acquired.

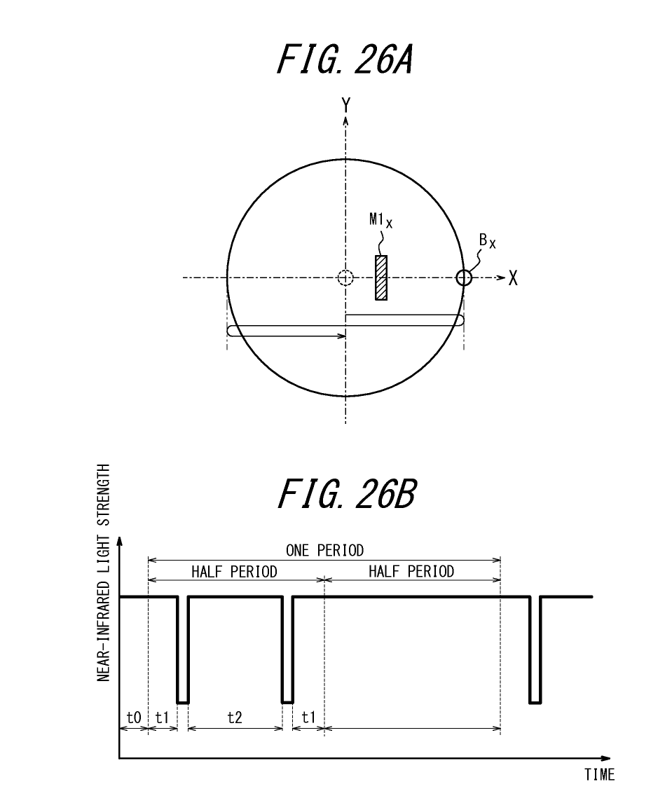

First, in the vibration adjustment mode, the laser 331IR of near-infrared light source is oscillated and drive frequency is sequentially varied so as to measure the amplitude variation of the emitting end 111c of the illumination optical fiber 111 in the X direction (Step S01). The method of measuring the amplitude is described in below with reference to FIGS. 26 and 27. First, the deflection magnetic field generating coils 281b, 281d cause the tip end part 111b of the illumination optical fiber 111 to vibrate in the X direction at a drive frequency to be measured, so as to irradiate near-infrared light to the object and detects the reflected light. FIG. 26 are views for illustrating an exemplary case where the light shielding mask 501 is used to optically scan the object at a predetermined amplitude before change over time, in which: FIG. 26A illustrates the light shielding region M1ₓ in the X direction on a lens surface on which the light shielding mask 501 is disposed and a scanning range of a scanning point Bx on the light shielding mask 501. In FIG. 26A, the light shielding region M1_{y} provided in the Y direction is omitted.

Light emitted from the illumination optical fiber 111 traverses the surface on which the light shielding mask 501 is disposed at a point in the X direction, the position of which substantially corresponding to the sine function of time. Near-infrared light transmitted through the surface having the light shielding mask 501 disposed thereon is partially reflected on the object 1001, condensed by the detection lens disposed in the fore stage of the detection optical fibers 121, passes through the detection optical fibers 121, converted into an electric signal by the photodetector 351, and output as a digital signal to the controller 311 by the ADC 361. Meanwhile, illumination light emitted from the illumination optical fiber 111 cannot be transmitted though the surface having the light shielding mask 501 disposed thereon, when traversing the light shielding region M1ₓ through the aforementioned X-direction scan, as being shielded by the light shielding region. In this case, a smaller signal is to be output to the controller 311. Based on the timing (time) when the output of near-infrared light is reduced, the controller 311 can calculate the amplitude of the emitting end 111c as described in below.

FIG. 26B shows the temporal change of signal strength of near-infrared light to be output to the controller 311 when a drive current of a sinusoidal wave is applied by the controller 311. Here, the time 0 corresponds to the time when the phase of the drive current becomes 0. Illumination light traverses twice the light shielding region M1ₓ during one period of vibration of the illumination optical fiber 111. The passage of light across the light shielding region M1ₓ can be detected as a substantially well-type decline in the output in the graph of FIG. 26B. In FIG. 26B, the time t1 corresponds to the time it takes for the illumination light to reach the light shielding region M1ₓ for the first time after passing through the origin point of the light shielding mask 501, and the time t2 corresponds to the time it takes for the illumination light having traversed the light shielding region M1ₓ to reach the maximum amplitude and returns to the light shielding region M1ₓ again. The time t1 is equal to the time it takes for the illumination light having traversed twice the light shielding region M1ₓ to reach the origin point for the first time. Here, the time t0 represents a phase delay of the vibration of the illumination optical fiber 111, with respect to the drive current generated by the actuator 211.

On the other hand, FIG. 27 are views for illustrating an exemplary case where the object is optically scanned at a narrower amplitude, using the same light shielding mask 501 as in FIG. 26, in which FIG. 27A illustrates the light shielding region M1ₓ in the X direction on a lens surface on which the light shielding mask 501 is disposed and a scanning range of the scanning point Bₓ on a surface where the light shielding mask 501 is disposed, and FIG. 27B indicates the temporal change of signal strength of near-infrared light to be detected. In this case, the amplitude of the vibration is narrowed, and thus, as illustrated in FIG. 27B, the ratio of t1 increases and the ratio of t2 decreases in one period of vibration, as compared with the case of FIG. 1. The scanning position in the X direction corresponds to the sine function with respect to time, and thus, the ratio between t1 and t2 may be measured so as to calculate the amplitude. Using this principle, the controller 311 can identify the amplitude in the X direction of the emitting end 111c of the illumination optical fiber 111. In Step S01, the drive frequency may gradually be varied within a predetermined range including the initial value of the resonance frequency, so as to measure the amplitude of the emitting end 111c of the illumination optical fiber 111.

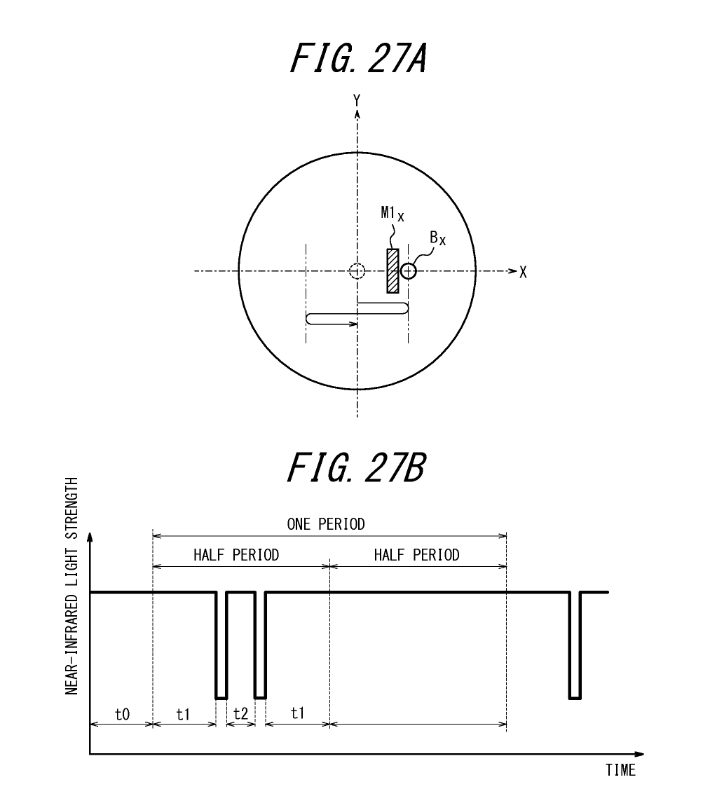

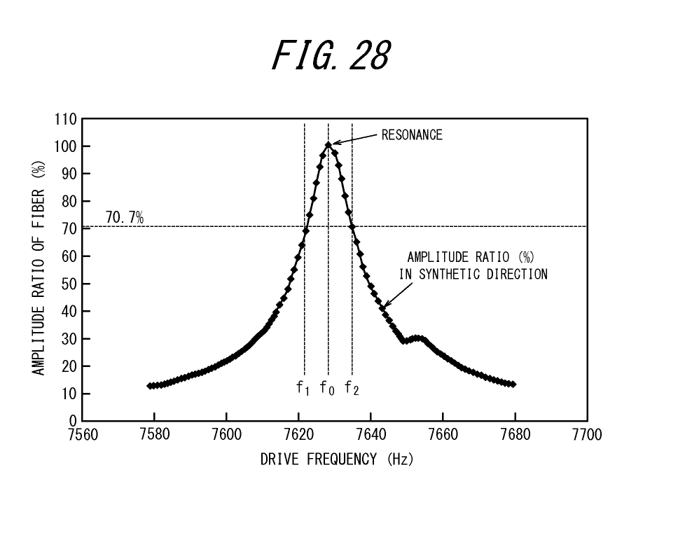

Next, the resonance frequency in the X direction and the Q value of the emitting end 111c of the illumination optical fiber 111 are calculated (Step S02). FIG. 28 is a graph showing the frequency characteristics of vibration, which is plotted with the amplitude ratio of the fiber with respect to the maximum value of the fiber amplitude of 100(%) being on the vertical axis and the drive frequency being on the horizontal axis. On this graph, the resonance frequency f₀ can be obtained as a drive frequency which gives the peak of the amplitude ratio of the fiber. Further, the Q value can be calculated from the expression below:

$$Q = \frac{{f}_{0}}{{f}_{2} - {f}_{1}} \qquad [ Expression 1 ]$$

where f₁ and f₂ each represent a drive frequency at which the amplitude ratio of the fiber becomes approximately 70.7% (=1/√2×100) of the resonance frequency f₀ on the low frequency side and on the high frequency side thereof.

After Step S02, as in the case of the X direction described above, the variation in amplitude relative to the drive frequency in the vicinity of the resonance frequency is measured also for the Y direction (Step S03), to thereby calculate the resonance frequency and the Q value (Step S04).

Here, the resonance frequency and the Q value of the fiber are known to have a large influence of the vibration orbit of the emitting end 111c of the illumination optical fiber 111. In light thereof, based on the resonance frequency and the Q value thus calculated, the drive current to be applied from the actuator 211 to the deflection magnetic field generating coils 281a to 281d, the drive frequency, and the phase thereof may be varied, to thereby adjust the vibration orbit of the emitting end 111c of the illumination optical fiber 111 so as to follow a predetermined orbit. In below, the principle thereof is described.

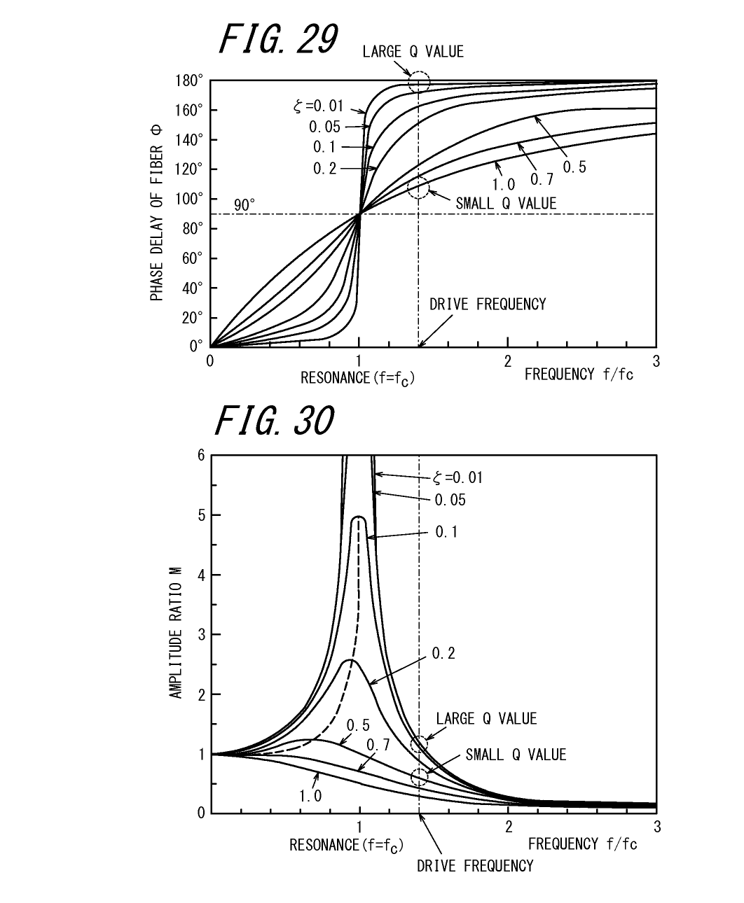

FIG. 29 is a graph showing a relation between the frequency of an optical fiber and the phase delay of the fiber. FIG. 30 is a graph for illustrating a relation between the frequency and the amplitude of an optical fiber. These graphs are publicly known in the field of wave engineering. In FIG. 29, a plurality of graphs are shown for each different ç. Here, ç is the damping ratio of the fiber vibration, which has the following relation with respect to the Q value:

$$Q = \frac{1}{2 + ζ} \qquad [ Expression 2 ]$$

In general, the Q value of the fiber vibration takes an extremely large value on the order of Q=50 to 300. Referring to FIG. 29, when a vibratory drive current is applied to the actuator 211, the fiber vibrates as delayed in phase, and the phase delay goes through a sudden change in the vicinity of the resonance frequency. Then, the change becomes steeper with the Q value being larger. FIG. 30 shows that the amplitude of the fiber rapidly increases in the vicinity of the resonance frequency, and the peak value becomes larger with the Q value being larger. The changes in phase delay appear as the change in t0 in FIGS. 26B and 27B.

Next, description is given of how to compensate the phase delay of the drive signal in the X direction and Y direction and how to calculate the magnitude of the drive current by the controller 311 (Step S05).

In scanning illumination light using an optical fiber, the drive frequency is generally set, not to the resonance frequency, but to a frequency slightly deviated therefrom. Referring to FIGS. 29, 30, the drive frequency is set to 1.4-fold (ω)/ω₀=1.4) of the resonance frequency. FIG. 29 shows that the change in value of ç causes the phase delay φ of the fiber to be shifted because graphs corresponding to different ç traverse ω/ω₀=1.4 at different points. Further, also in the case where the resonance frequency (f_{c}) is shifted, the drive frequency with respect to the resonance frequency (f_{c}) relatively changes, causing the phase delay φ of the fiber to be shifted. Meanwhile, according to FIG. 30, the amplitude ratio Mat the drive frequency (ω/ω₀=1.4) changes in accordance with the change in Q value. Further, when the resonance frequency (f_{c}) is shifted, the drive frequency with respect to the resonance frequency (f_{c}) also varies, causing the amplitude ratio M to change.

When the resonance frequency and the Q value of the tip end part 111b of the illumination optical fiber 111, and the drive power of the actuator 211 for driving the deflection magnetic field generating coils 28a to 28d are changed due to change over time so as to generate a slight difference in the amount of change in the X direction and Y direction, the amplitude and the phase in the X direction and the Y direction suffer a great change, leading to distortion to be generated in the vibration trajectory of the emitting end 111c of the illumination optical fiber 111. Meanwhile, the phase delay and the amplitude ratio of the fiber can be logically calculated from the resonance frequency and the Q value as shown in the graphs of FIGS. 29 and 30. Thus, the controller 311 calculates the phase delay t0 of the fiber, based on the resonance frequency and the Q value in the X direction and the Y direction thus calculated, and determines the compensation amount of phase of a current to be applied to the deflection magnetic field generating coils 281a to 281d of the actuator 211, so as not to cause any change in phase of the emitting end 111c of the illumination optical fiber 111 in the X direction and the Y direction from before the change over time. Further, the controller 311 sets the magnitude of current to be applied to the deflection magnetic field generating coils 281a to 281d of the actuator 211, such that the amplitude of current constantly remains the same in the X direction and in the Y direction and a desired amplitude can be obtained.

Next, in the image acquisition mode, visible light lasers 331R, 331G, and 331B are oscillated, and applies the compensation amount in the X direction and the Y direction and the drive current (maximum amplitude value) calculated in Step S05, so as to carry out scanning in a spiral manner (spiral scan) which is reduced in distortion on the object. Visible light signals of different colors obtained by the irradiation of laser light onto the object 1001 are, as has been already explained, propagated through the detection optical fibers 121 so as to be split into spectral component by the photodetector 351, converted into digital signals by the ADC 361, and output to the image processor 371. In the case of spiral scan, the amplitude is gradually changed from 0 to the current value calculated in Step S05, so as to take in an image for one frame (Step S06).

The controller 311, after acquiring a one-frame image, repeats again the vibration adjustment mode (Steps S01 to S05) and the image acquisition mode (Step S06), unless instructed to stop image acquisition (Step S07).

As described above, according to Embodiment 3, the light shielding mask 501 is provided so as to spatially and partially reduces the transmission of near-infrared light included in light emitted from the emitting end 111c, and the timing (time interval) at which the near-infrared light is shielded by the light shielding regions M1ₓ, M1_{y} on the light shielding mask 501 is identified based on the electric signal detected by the photodetector 351, to thereby calculate, based on the signal, the resonance frequency and the Q value changed over time. Those drive parameters may be used to correct the drive conditions of the actuator 211 such that the vibration of the emitting end 111c of the illumination optical fiber 111 returns to the state before the change over time, which allows for obtaining a stable image with no distortion or reduced distortion.

Further, according to Embodiment 3, the drive conditions in the vibration adjustment mode are corrected every time a one-frame image is acquired in the image acquisition mode, which allows for understanding in real time the scanning state even when the scanning trajectory on the object 1001 is changed along with time due to the change in environmental conditions during use, and also for correcting the drive conditions during the use of the apparatus without the need for complicated work, so as to return the changed scanning trajectory to the original scanning trajectory.

Further, Embodiment 3 is also advantageous in that it is only necessary to provide the light shielding mask 501 of the optical system 251 of the scope 201, the laser 331IR of the control 301, and other elements such as a demultiplexer for demultiplexing near-infrared light and a detecting element for detecting near-infrared light within the photodetector 351, without the need for additionally providing a complicated apparatus.

Further, the wavelength of near-infrared light is set so as not to coincide with the absorption wavelength, which allows for the detection of high contrast. Thus, the resonance frequency can be detected with ease.

Here, according to Embodiment 3, the vibration adjustment mode is to be carried out for each one-frame image acquisition, before and after the one-frame image acquisition, but not limited thereto and may be carried out at various timings. For example, the vibration adjustment mode may be carried out only once at every activation of the fiber scanning endoscope apparatus 101. Alternatively, the mode may be carried out at ever lapse of a predetermined time such as 10 minutes, 1 hour after the activation of the device.

Further, the light shielding mask 501 is provided on a plane of the plano-concave lens 251d disposed in the optical system 251 at a position closest to the object 1001, but not limited thereto. For example, a light shielding mask may be disposed between the plano-convex lenses 251a and 251b.

Further, in Embodiment 3, the drive conditions such as the amplitude and the phase of a current to be applied to the actuator 211 are varied based on the amplitude, the resonance frequency, and the Q value in each of the X direction and the Y direction. However, the image distortion resulting from the phase shift may be compensated by the image processor 371 through correcting the acquired image signal.

### Embodiment 4

Embodiment 4 assumes a case where, in the fiber scanning endoscope apparatus 101 of Embodiment 3, a current value to be applied to the actuator 211 is varied without changing the predetermined drive frequency when the variation of the Q value is negligible small and the change of the amplitude is not so great, unlike in Embodiment 3 in which the drive frequency is varied within a predetermined frequency range so as to measure the change in amplitude.

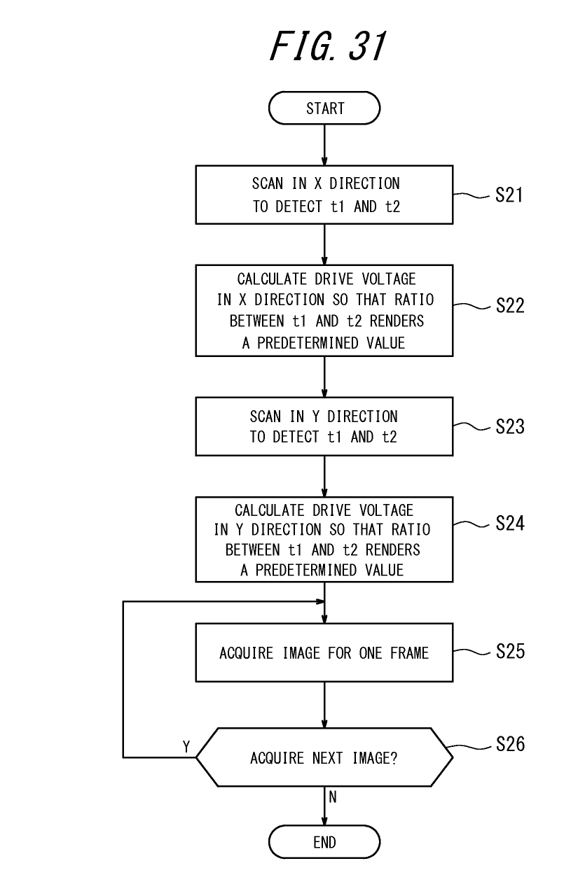

FIG. 31 is a flowchart illustrating an image acquisition procedure according to Embodiment 4. Similarly to the case described in Embodiment 3 with reference to FIG. 26, the actuator 211 drives the tip end part 111b of the illumination optical fiber 111 in the X direction, so as to acquire the time t1 corresponding to the time it takes for the illumination light to reach the light shielding region M1ₓ for the first time after passing through the origin point on a plane where the light shielding mask 501 is disposed, and the time t2 corresponding to the time it takes for the illumination light having traversed the light shielding region M1ₓ to reach the maximum amplitude and returns to the light shielding region M1ₓ again (Step S21). At this time, the drive frequency is set to a value yet to be changed over time (such as a design value for the product, or a value calibrated by other method immediately before). Next, based on the times t1 and t2 thus acquired, the drive current value in the X direction to be applied to the actuator 211 is calculated so that the ratio between t1 and t2 renders a predetermined value yet to be changed over time (Step S22). Similarly, the time t1 and t2 are acquired for the Y direction (Step S23), and the drive current value in the Y direction to be applied to the actuator 211 is calculated (Step S24).

Using the drive current in each of the X direction and the Y direction thus calculated, a one frame image is acquired in the image acquisition mode (Step S25). Thereafter, the acquisition of a one-frame image is repeated (Step S25), unless an instruction to stop the image acquisition is received (Step S26). Note that, similar to Embodiment 3, the vibration adjustment mode (Step S21 to S24) and the image acquisition mode (Step S25) may be alternately carried out, or the vibration adjustment mode may be carried out at any other timing.

According to Example 4, scanning is performed in the X direction and the Y direction at least once, so as to measure the intervals t1, t2 of time traversing the light shielding regions M1ₓ, M1_{y}, based on which a current value to be applied to the actuator 211 is corrected, and thus the phase delay associated with the displacement of the resonance frequency and the Q value cannot be compensated. However, unlike in Embodiment 3, there is no need to repeatedly measure the amplitude by changing the frequency within a predetermined frequency range, and thus the amplitude of the emitting end 111c of the illumination optical fiber 111 can be corrected in a short period of time, and the control method can be simplified.

Although the phase delay cannot be compensated in Embodiment 4 as describe above, the drive signal to be applied to the actuator by the controller 311 and the strength signal detected by the photodetector 351 may be synchronized, to thereby estimate to. In this case, the phase of the drive signal may be adjusted in such a manner as to compensate the estimated phase delay in the X direction and the Y direction, so that not only the change of the scanning amplitude over time but also the scanning waveform distortion resulting from the phase shift can be corrected. In this case as well, unlike in Embodiment 3, there is no need to carry out a number of amplitude measurement by sequentially changing the frequency so as to calculate the resonance frequency or the Q value, which means that the processing can be performed in a short period of time.

### Embodiment 5

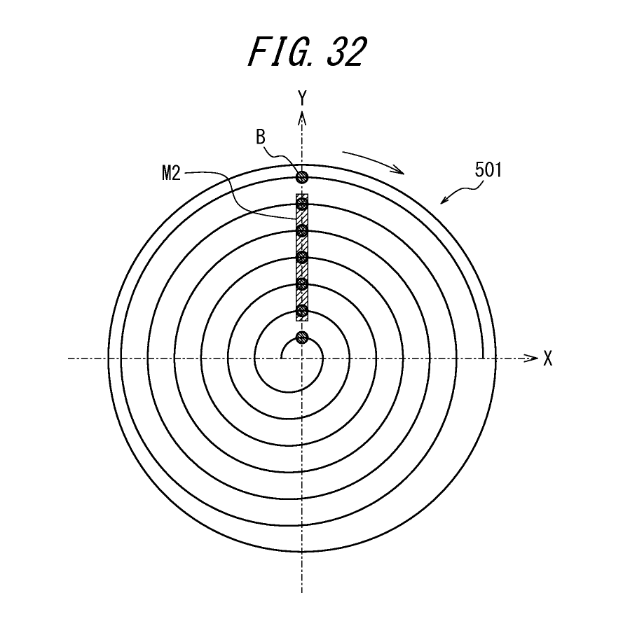

FIG. 32 is for illustrating a light shielding mask 501 according to Embodiment 5 and a scanning trajectory of illumination light traversing on a plane where the light shielding mask 501 is disposed. The fiber scanning endoscope apparatus 101 of Embodiment 5 spirally scans the object 1001. The light shielding mask 501 is disposed on the object 1001 side plane of the plano-concave lens 251d in the optical system 251 of the scope 201, as in Embodiment 3. The light shielding mask 501 has a light shielding region M2 that is rectangular in shape elongated in the Y direction, but the light shielding region M2 does not extend in the vicinity of the origin point (center of spiral scan) and near the outer circumferential part of spiral scan. The rest of the configuration is similar to that of Embodiment 3, and thus, the same constituent elements are denoted by the same reference symbols to omit the description thereof.

Next, description is given of the operation of the fiber scanning endoscope apparatus 101 of Embodiment 5. Unlike in Embodiments 3 and 4, the compensation of the scanning trajectory by the scanning of near-infrared light and the image acquisition of an object through the irradiation of visible laser are carried out at the same time. In the spiral scan, illumination light including near-infrared light and visible light traverses once the light shielding region M2 every one round made by the scanning point B of the illumination light, except when scanning the vicinity of the scan center and the outer circumferential part.

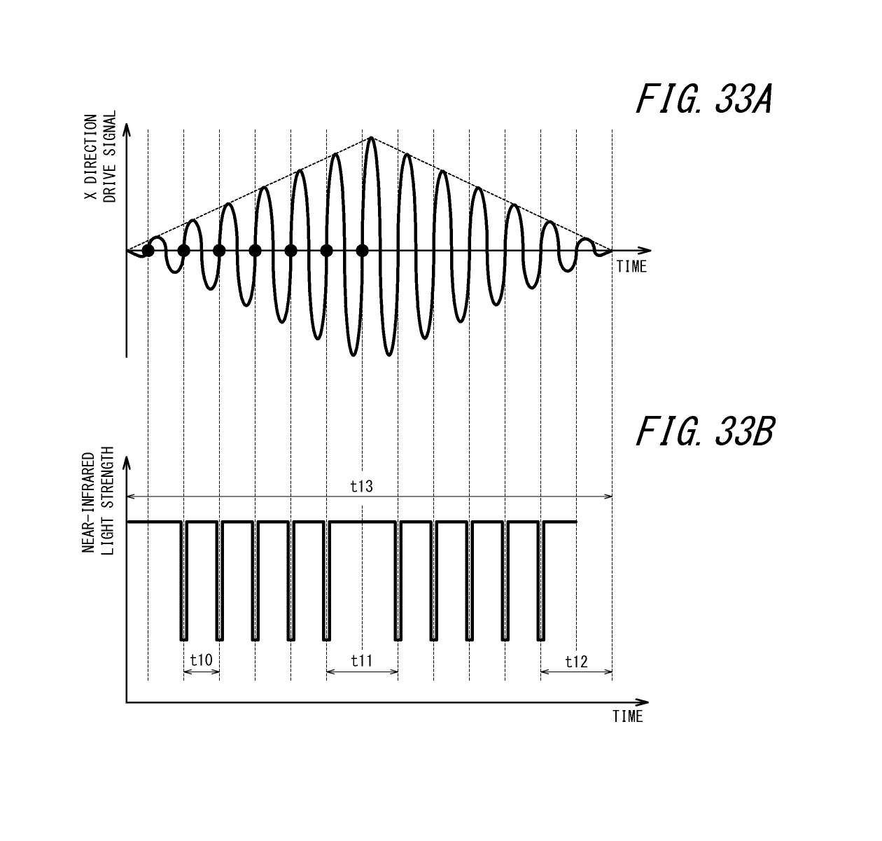

FIGS. 33A and 33B are graphs for illustrating a method for analyzing the frequency and the amplitude for each round of spiral scan using the light shielding mask of FIG. 32, where FIG. 33A shows a drive signal in the X direction to be applied to the actuator 211, and FIG. 33B shows a temporal change of signal strength of near-infrared light to be detected. Based on the interval t10 of timings at which a well-type decline is generated in the output of the signal strength of infrared light shown in FIG. 33B, the period (frequency and phase) of each round can be calculated. In spiral scan, the period of each round may be slightly shifted along with fluctuations in the amplitude. Such shift generated in the period causes a phase shift in an image to be acquired. For example, there is a fear that pixel data acquired at a timing of detecting a point on the Y axis should actually correspond to another point displaced in the circumferential direction from the intended point on the Y axis. In such case, the coordinate to be identified by the controller 311 based on the time elapsed after the drive start is inadvertently shifted from the actually-scanned position on the object 1001, with the result that an acquired image will be significantly distorted.

Further, the time t11 during which the illumination light irradiated does not traverse the light shielding region M2 in the outer circumferential part may be measured, so as to calculate the maximum amplitude of the spiral orbit. For example, when the amplitude of spiral scan follows a pattern shown in FIG. 33A which increases from zero in proportional to time to reach a maximum value and then decreases along with time, the maximum amplitude of spiral scan is larger as the ratio of time t11 with respect to the period t13 of one spiral scan is higher, and thus, the maximum amplitude can readily be calculated.

Further, visible light is irradiated onto the object 1001 simultaneously with detecting the change of the scanning orbit over time through the irradiation of near-infrared light, which allows for acquiring a color image of one frame through one spiral scan. In the next frame, the controller 311 changes the drive current to be applied to the actuator 211, based on the measured information on t11, so as to adjust the maximum amplitude of spiral scan, more properly, the amplitude in the Y direction not to change from a desired value. Further, based on the measured data on t10, the controller 311 compensates the positional shift in the circumferential direction of the pixel data acquired through spiral scan by the image processor 371, so as to generate image data, to thereby reduce distortion in the acquired image.

As described above, according to Embodiment 5, the period and the maximum amplitude of the scan can be detected simultaneously with the acquisition of image data on the object 1001 through spiral scan. This configuration allows for correcting distortion in the acquired image and maintaining the scanning range constant. Further, there is no need to suspend the acquisition of image data in order to correct the drive conditions, and thus images can be acquired at high frame rate.

In Embodiment 5, the light shielding mask 501 is rectangular in shape extending in the Y direction, but the shape of the light shielding mask 501 is not limited thereto. The light shielding region of the light shielding mask may be formed in various shapes such as a dot, a line, a curved line, and a regional. FIG. 34 each illustrate a variation of the light shielding mask, where FIG. 34A shows a light shielding mask having dot-shaped light shielding regions M3 distributed; FIG. 34B shows another light shielding mask having a plurality of linear-shaped light shielding regions M4, and FIG. 34C shows still another light shielding mask having a semicircular light shielding region M5. For example, in the light shielding mask of FIG. 34A, the light shielding regions M3 are distributed in both of the X direction and the Y direction, and thus, the amplitudes in the X direction and the Y direction can both be measured. Further, in the light shielding mask of FIG. 32, the amplitude cannot be measured when the amplitude is smaller than the distance between the end on the outer circumferential side of the light shielding region M2 and the scan center; whereas the light shielding regions M3 are distributed as spaced apart from each other, which makes it possible to measure the amplitude in a wider range. Further, in the light shielding mask of FIG. 34B, the linear light shielding regions are each arranged at an angle associated with the distance from the center. Further, in the light shielding mask of FIG. 34C, the light shielding region M5 covers the half of the circular scanning region. Such light shielding masks are simple in shape and easy to form.

### Embodiment 6

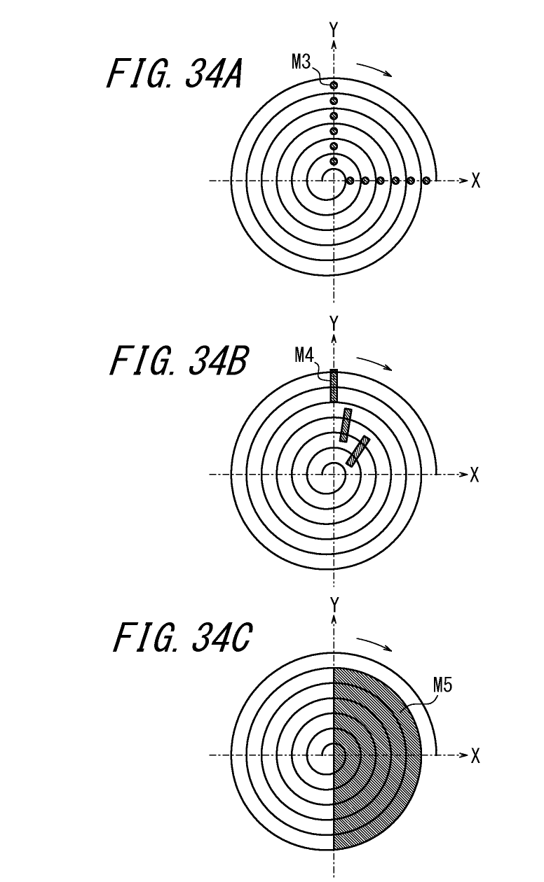

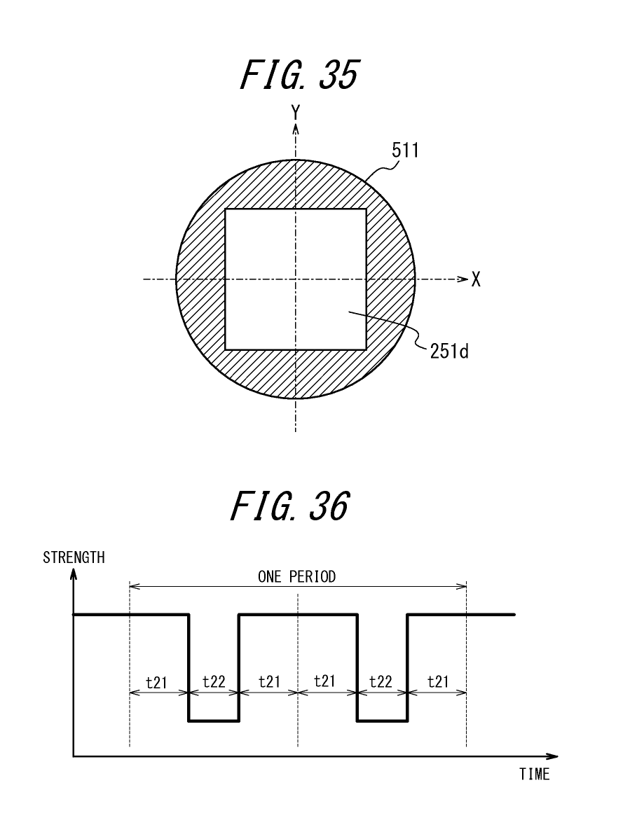

FIG. 35 is a front view (in a direction where the illumination light is emitted) of the mirror frame 511 and the lens 251d located at the scope tip of a fiber scanning endoscope apparatus according to Embodiment 6. The mirror frame 511 is provided to hold the plano-convex lenses 251a, 251b and the plano-concave lens 251c, 251d of the optical system 251. In Embodiment 6, unlike in Embodiment 3, no light shielding region is provided on the object 1001 side plane of the plano-concave lens 251d. Instead, the mirror frame 511 is in a shape having a square opening in the center, and thus, part of illumination light emitted from the illumination optical fiber 111 that passes through an optical path at a distance from the optical axis is shielded by the mirror frame 511. In other words, the mirror frame 511 serves as an light reducing part for shielding some of the light beams passing through the plano-concave lens 251d.

On the other hand, as compared with Embodiment 3, Embodiment 5 does not necessitate the laser 331IR as a near-infrared light source and a near-infrared light detecting element of the photodetector 351 provided correspondingly to the laser 331IR. The rest of the configuration is similar to that of Embodiment 3, and thus, the same constituent elements are denoted by the same reference symbols to omit the description thereof.

With the tip end part 241 of the scope 201 configured as described above, in the vibration adjustment mode, either one of the lasers 331R, 331G, 331B are oscillated so as to vibratory drive the tip end part 111b of the illumination optical fiber 111 in the X direction, allowing a planar lens surface at the tip of the plano-concave lens 251d to be scanned. At this time, the amplitude in the X direction becomes larger, light emitted from the emitting end 111c of the illumination optical fiber 111 hits the mirror frame 511 to be shielded. Therefore, only light that has passed through the plano-concave lens 251d without being shielded by the mirror frame 511 reaches the object 1001 to be reflected in part, so as to pass through the detection optical fibers 121 to be detected by the photodetector 351 of the control main body 301.

FIG. 36 is a graph showing a temporal change of signal light strength to be detected when visible light is scanned in the X direction using the mirror frame of FIG. 35. The graph shows time t21 it takes for the light to traverse the end of the mirror frame 511 from the origin point, and time t22 during which the light is shielded by the mirror frame 511. As in Embodiment 3, the amplitude may be calculated by detecting t21 and t22, and further, the resonance frequency and the Q value may also be calculated.

Therefore, according to Embodiment 6, there may similarly be obtained, as in Embodiment 3, information on the change of the amplitude over time, the resonance frequency, and the Q value that may change the vibration waveform of the emitting end 111c of the illumination optical fiber 111, without using any near-infrared light source and any detection element therefor. This way allows the drive conditions of the actuator 211 to be changed so as to compensate the influence resulting from the change of the scanning orbit over time, to thereby obtain a stable image with no distortion or reduced distortion.

It should be noted that the present disclosure is not limited only to Embodiments 3 to 6 described above, and may be subjected to various modifications and alterations. For example, in Embodiments 3 to 6 above, the actuator is configured as an electromagnetic drive means using a permanent magnet and electromagnetic coils, but not limited thereto. For example, a drive means using a piezoelectric element may also be used.

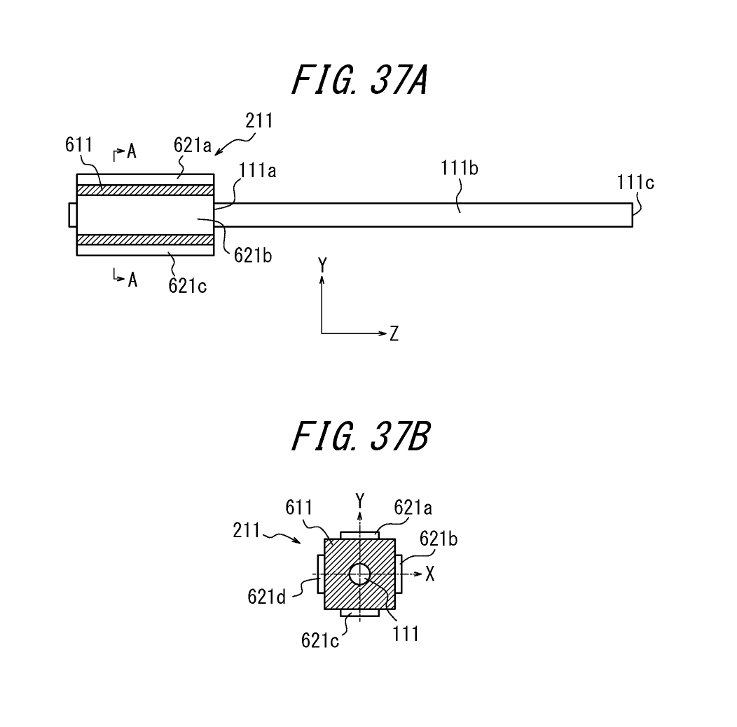

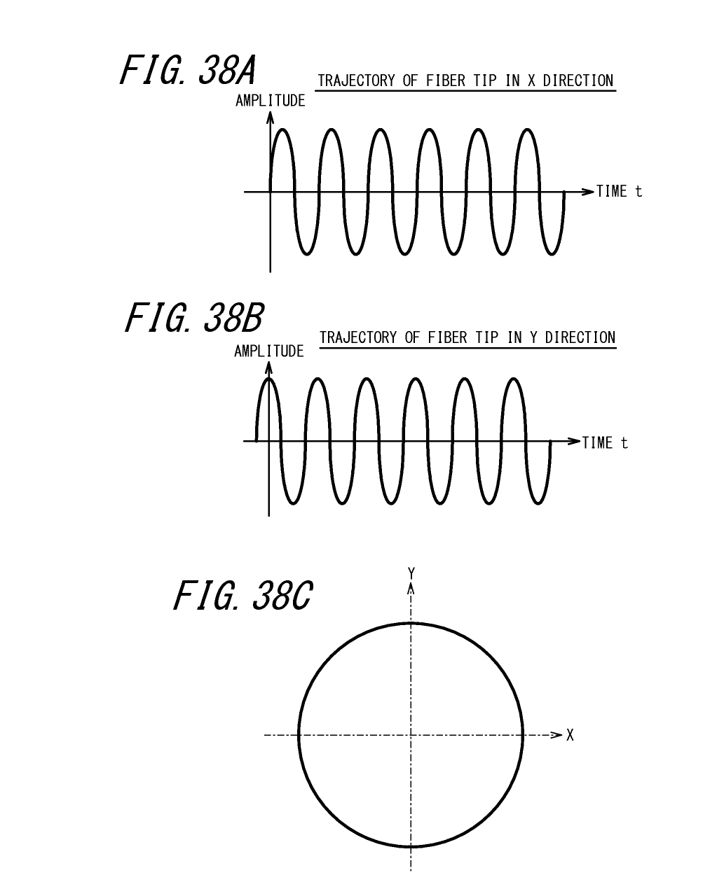

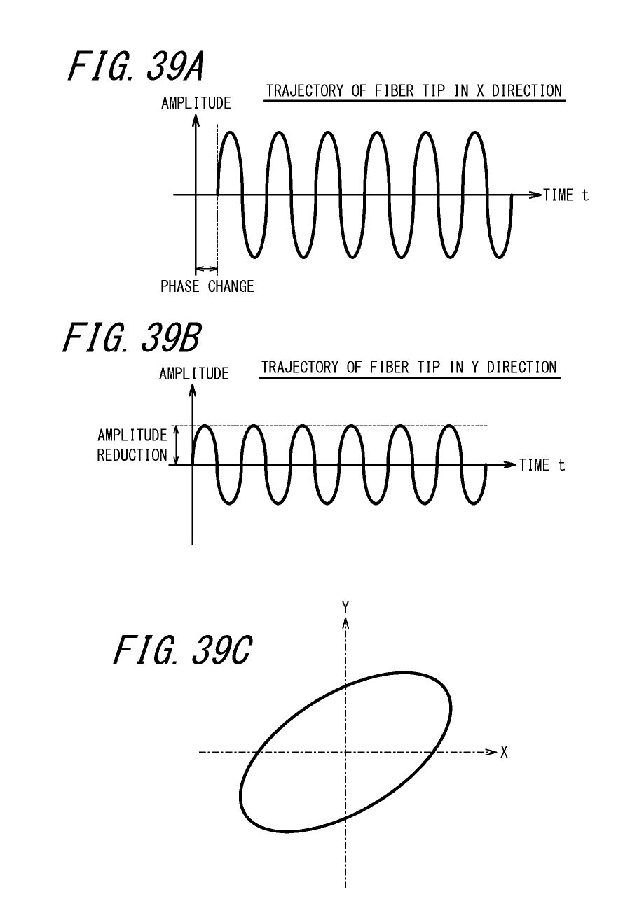

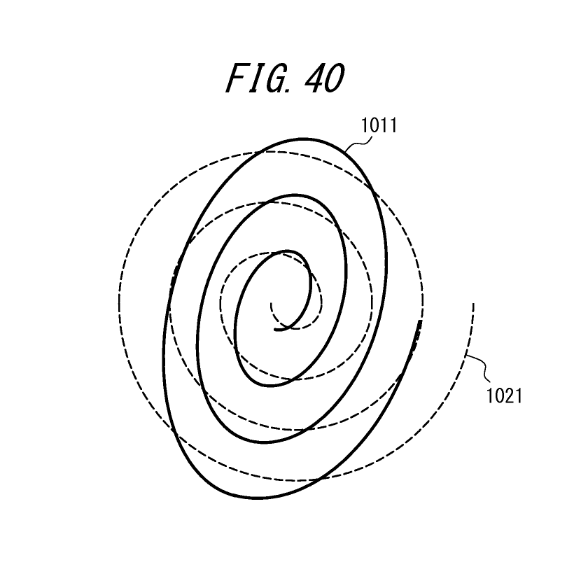

FIG. 37 each illustrate an example where the actuator 211 in Embodiments of the subject application is configured by using a piezoelectric element. FIG. 37A is a side view of the actuator 211, and FIG. 37B is a sectional view taken along the line A-A of FIG. 37A. The illumination optical fiber 111 passes through the center of a fiber holding member 611 having an angular pillar shape formed of an elastic material, so as to be fixed and held by the fiber holding member 611. The four side surfaces of the fiber holding member 611 each faces in the +Y direction and the +X direction and the directions opposite thereto. Then, a pair of Y-direction drive piezoelectric elements 621a, 621c are fixed to the fiber holding member 611 respectively in the +Y direction and the −Y direction, and a pair of X-direction drive piezoelectric elements 621b, 621c are fixed to the fiber holding member 611 respectively in the +X direction and the −X direction.

Connected to each of the piezoelectric elements 621a to 621d is the wiring cable 131 from the drive controller 381 of the control main body 301. In order to drive the piezoelectric elements 621a to 621d of the actuator 211, the drive controller 381 receives a control signal from the controller 311 and generates a drive voltage in the X direction and in the Y direction, respectively, so as to drive via the wiring cable 131 the piezoelectric elements 621a to 621d disposed at the tip end part 241 of the scope 201.

More specifically, voltages applied between the piezoelectric elements 621b and 621d in the X direction always have opposite polarity but are equal to each other in magnitude. Similarly, voltages applied between the piezoelectric elements 621a and 621c in the Y direction always have opposite polarity but are equal to each other in magnitude. The piezoelectric elements 621b, 621d disposed as opposed to each other across the fiber holding member 611 are mutually contract and extend in a trade-off relation so as to cause deflection in the fiber holding member 611, and such contraction and extension are alternately repeated, to thereby cause vibration in the X direction. The same applies to the vibration in the Y direction.

The disclosed apparatus according to Embodiments may produce the same operation and effect even when the actuator 211 is replaced with the configuration of FIG. 37 that uses the piezoelectric elements 621a to 621d.

Further, the disclosed apparatus can also be applied to any other scanning waveform than spiral scan. In the case of raster scan, one of the two orthogonal scanning directions scans in the vicinity of the resonance frequency, and thus the compensation of the amplitude and the phase according to the present disclosure is effective. The present disclosure may be applied not only to the fiber scanning endoscope apparatus but also other applications such as fiber scanning microscope.

1. An optical scanning unit comprising:
an optical fiber having an emitting end thereof oscillatably supported, the emitting end being oscillated while irradiating an object with light emitted from the emitting end, to thereby scan the object;
an actuator for oscillating the emitting end;
a reducing part disposed at a predetermined position relative to a designed optical axis of the optical fiber when not oscillated, the predetermined position being on the object side relative to the emitting end, the reducing part reducing the transmission of light having at least part of the bandwidth of light emitted from the emitting end;
a detection part for detecting light at the object when light emitted from the emitting end is irradiated onto the object; and
a controller for calculating, in an image formed based on the light detected by the detection part and the state of oscillation of the optical fiber, the eccentricity between the designed optical axis and the actual optical axis of the optical fiber, based on a position where the received amount of the light having part of the bandwidth is reduced and the center position of the image.

2. The optical scanning unit according to claim 1, wherein the reducing part reduces the transmission of at least any of light of three colors minimally necessary for forming the image as a color image.

3. The optical scanning unit according to claim 2, wherein the controller calculates the eccentricity in an eccentricity calculation frame.

4. The optical scanning unit according to claim 3, wherein the eccentricity calculation frame is carried out at least any one of: in between main purpose frames for scanning the object for a different purpose rather than to calculate the eccentricity; and before or after an operation mode for scanning the object for the different purpose.

5. The optical scanning unit according to claim 1, wherein the reducing part transmits light of three colors necessary for forming the image as a color image, and reduces transmission of light of different bandwidth from the light of three colors.

6. The optical scanning unit according to claim 1, wherein the reducing part substantially shields light having at least part of the bandwidth of light emitted from the emitting end.

7. The optical scanning unit according to claim 1, wherein the reducing part reduces the transmission of the light having part of the bandwidth, in a region capable of passing or transmitting light.

8. The optical scanning unit according to claim 1, wherein the reducing part is in a shape that surrounds around the region capable of passing or transmitting light.

9. The optical scanning unit according to claim 8, further comprising an illumination optical system disposed in the emission direction of the emitting end,
wherein the reducing part is disposed on a mirror frame holding the illumination optical system.

10. The optical scanning unit according to claim 8, further comprising an illumination optical system disposed in the emission direction of the emitting end,
wherein the reducing part is formed as a film for reducing the transmission of the light having part of the bandwidth, the film being disposed on a surface of an optical element included in the illumination optical system.

11. The optical scanning unit according to claim 8, wherein the region capable of passing or transmitting light within the reducing part is circular or rectangular in shape.

12. The optical scanning unit according to claim 8, wherein the controller causes:
the actuator to oscillate the emitting end such that, when calculating the eccentricity, light emitted from the emitting end is irradiated onto a region larger than the inside of the reducing part; and
the actuator to oscillate the emitting end such that, at times other than calculating the eccentricity, light emitted from the emitting end is irradiated onto a region smaller than the inside of the reducing part.

13. The optical scanning unit according to claim 1, further comprising an illumination optical system disposed in the emission direction of the emitting end,
wherein the reducing part is disposed on the object side relative to the incident pupil position of the illumination optical system.

14. The optical scanning unit according to claim 1, wherein the controller oscillates the emitting end in a state where the eccentricity of the optical fiber has been compensated based on the eccentricity calculated.

15. The optical scanning unit according to claim 1, wherein the actuator is a piezoelectric actuator or an electromagnetic actuator.

16. An optical scanning observation apparatus including the optical scanning unit according to claim 1.

17. An optical fiber scanning apparatus, comprising:
an optical fiber for guiding light from a light source and irradiating the light onto an object;
an actuator for vibratory driving a tip end part of the optical fiber;
a light reducing part for partially reducing transmission of light having at least part of the bandwidth of light emitted from an emitting end of the tip end part;
a detection part for detecting light to be detected, the light being obtained from the object, through irradiation of light emitted from the emitting end; and
a controller for controlling the vibratory-drive of the actuator,
wherein the controller identifies, based on a signal detected by the detection part, timing for reducing, by the light reducing part, the transmission of the light having at least part of the bandwidth.

18. The optical fiber scanning apparatus according to claim 17, wherein the controller calculates, based on the timing for reducing the transmission of the light having at least part of the bandwidth, the amplitude of the tip end part of the optical fiber.

19. The optical fiber scanning apparatus according to claim 18,
wherein the actuator sequentially changes, within a predetermined frequency range, the drive frequency for vibratory driving the tip end part of the optical fiber, and
wherein the controller calculates, based on the drive frequency and the calculated amplitude of the tip end part, the resonance frequency of the tip end part of the optical fiber.

20. The optical fiber scanning apparatus according to claim 19, wherein the controller calculates, based on the drive frequency in sequence and the calculated amplitude of the tip end part, the Q value of the vibration of the tip end part of the optical fiber.

21. The optical fiber scanning apparatus according to claim 17,
wherein the actuator is capable of vibratory driving the tip end part of the optical fiber individually in at least two driving directions, and
wherein the controller calculates, for each of the at least two driving directions, the amplitude of the tip end part of the optical fiber.

22. The optical fiber scanning apparatus according to claim 17, wherein the controller calculates the amplitude of the drive signal to be applied to the actuator such that the time interval of the timing for reducing the transmission of the light having at least part of the bandwidth is made coincide with a predetermined time interval.

23. The optical fiber scanning apparatus according to claim 17, wherein the controller changes, based on information obtained from the timing for reducing the transmission of the light having at least part of the bandwidth, either one or both of the amplitude and the phase of the drive signal to be applied to the actuator, so as to keep constant the trajectory of the emitting end of the optical fiber.

24. The optical fiber scanning apparatus according to claim 17, further comprising an image acquisition part for acquiring image data on the object, and having a vibration adjustment mode and an image acquisition mode,
wherein, in the vibration adjustment mode, the controller vibratory drives the actuator, and calculates, based on the timing for reducing the transmission of the light having at least part of the bandwidth, a correction value of the drive parameter of the actuator including at least one of the amplitude and the phase of the drive signal to be applied to the actuator in order for obtaining a predetermined vibration trajectory, and
wherein, in the image acquisition mode, the controller vibratory drives the actuator based on the correction value of the drive parameter, and the image acquisition part acquires image data on the object, from the signal detected by the detection part.

25. The optical fiber scanning apparatus according to claim 24, wherein the vibration adjustment mode is carried out, for each image acquisition for one frame in the image acquisition mode, before and after the image for one frame is acquired.

26. The optical fiber scanning apparatus according to claim 24, wherein the vibration adjustment mode is carried out after activation of the scanning detection apparatus and before executing the image acquisition mode.

27. The optical fiber scanning apparatus according to claim 17, further comprising an optical system for irradiating, toward the object, light emitted from the emitting end of the optical fiber,
wherein the light reducing part is provided to a mirror frame holding the optical system.

28. The optical fiber scanning apparatus according to claim 17, further comprising an optical system for irradiating, toward the object, light emitted from the emitting end of the optical fiber,
wherein the light reducing part is formed on a surface of an optical element constituting the optical system.

29. The optical fiber scanning apparatus according to claim 17, wherein the light reducing part is selected so as to reduce the transmission of light having a wavelength that does not coincide with the absorption wavelength of the object.

30. An optical fiber scanning apparatus, comprising:
an optical fiber having a tip end part oscillatably supported, the optical fiber guiding light from a light source to irradiate an object with the light;
an actuator for vibratory driving the tip end part of the optical fiber;
a light reducing part for partially reducing transmission of light having at least part of the bandwidth of light emitted from an emitting end of the tip end part;
a detection part for detecting light to be detected, the light being obtained from the object through irradiation of light emitted from the emitting end; and
a controller for controlling the vibratory-drive of the actuator,
wherein the controller identifies, based on a signal detected by the detection part, timing for reducing, by the light reducing part, the transmission of the light having at least part of the bandwidth, and calculates, based on the timing, a vibration period of each vibration of the tip end part of the optical fiber.

31. The optical fiber scanning apparatus according to claim 30, wherein the controller vibratory drives the actuator in a two-dimensional direction so as to two-dimensionally scan the object.

32. The optical fiber scanning apparatus according to claim 31, wherein the two-dimensional scan is spiral scan.

33. The optical fiber scanning apparatus according to claim 32,
wherein the light reducing part is disposed so as to radially traverse, on the optical path of the spiral scan, a region that does not include the scan center and an outermost circumferential part of the spiral scan, and
wherein the controller calculates, based on the timing for reducing the transmission of the light having at least part of the bandwidth, the amplitude of the tip end part of the optical fiber.

34. The optical fiber scanning apparatus according to claim 33, further comprising an image acquisition part for acquiring image data on the object,
wherein the image acquisition part acquires image data on the object from the signal detected by the detection part, through each scan of the object, and
wherein the controller calculates the period and the amplitude of each vibration of the tip end part, and adjusts, based on the amplitude and the period thus calculated, a drive signal to be applied to the actuator.
